(19) United States
(12) Patent Application Publication
(10) Pub. No.: US 2019/0022371 A1
(43) Pub. Date: Jan. 24, 2019

(54) ELECTRODE ARRAY FOR TRANSCUTANEOUS ELECTRICAL STIMULATION OF THE SPINAL CORD AND USES THEREOF

(71) Applicant: The Regents of the University of California, Oakland, CA (US)

(72) Inventors: Chih-Wei Chang, Los Angeles, CA (US); Victor Reggie Edgerton, Los Angeles, CA (US); Wentai Liu, Los Angeles, CA (US); Yuri Gerasimenko, Los Angeles, CA (US); Parag Gad, Woodland Hills, CA (US); Yi-Kai Lo, Los Angeles, CA (US)

(21) Appl. No.: 15/750,499

(22) PCT Filed: Aug. 5, 2016
(86) PCT No.: PCT/US2016/045898

§ 371 Date: Feb. 5, 2018

(51) Int. Cl. A61N 1/04; A61N 1/05; A61L 31/02; A61N 1/08; A61N 1/36

(52) U.S. Cl. CPC A61N 1/0456; A61N 1/0502; A61N 1/36; A61N 1/08; A61L 31/02

(57) ABSTRACT

In various embodiments, a transcutaneous needle electrode and uses thereof are provided. In certain embodiments the needle electrodes comprise a plurality of electrically conductive needles, where the needles are solid, or where the needles are hollow and have a closed tip, where the needles have an average tip diameter less than about 10 μm and an average length greater than about 20-50 μm wherein the electrically conductive needles are electrically coupled to one or more electrical leads.

### CROSS-REFERENCE TO RELATED APPLICATIONS

This application claims benefit of and priority to U.S. Ser. No. 62/201,973, filed on Aug. 6, 2015, which is incorporated herein by reference in its entirety for all purposes.

### STATEMENT OF GOVERNMENTAL SUPPORT

[Not Applicable]

### BACKGROUND

Serious spinal cord injuries (SCI) affect approximately 1.3 million people in the United States, and roughly 12-15,000 new injuries occur each year. Of these injuries, approximately 50% are complete spinal cord injuries in which there is essentially total loss of sensory motor function below the level of the spinal lesion.

Neuronal networks formed by the interneurons of the spinal cord that are located in the cervical and lumbar enlargements, such as the spinal networks (SNs), can play an important role in the control of posture, locomotion and movements of the upper limbs, breathing and speech. Normally, the activity of SNs is regulated supraspinally and by peripheral sensory input. In the case of disorders of the connections between the brain and spinal cord, e.g., as a result of traumatic spinal cord lesions, motor tasks can be enabled by epidural and transcutaneous electrical stimulation of the lumbosacral and cervical segments of the spinal cord as well as the brainstem.

However, the use systems to provide transcutaneous electrical stimulation has been hampered by the necessity to deliver relatively high voltage stimulation at the skin surface often resulting in discomfort and/or irritation and reduced subject compliance.

### SUMMARY

In various embodiments a novel needle (microneedle) electrode is provided that is suitable for transcutaneous electrical stimulation of the spinal cord. The needle electrodes described herein are well suited for transcutaneous electrical stimulation with low impedance and conformal electrical field distribution.

Various embodiments contemplated herein may include, but need not be limited to, one or more of the following:

### EMBODIMENT 1

A needle electrode for transcutaneous neural stimulation, said electrode including: a plurality of electrically conductive needles, wherein said needles are solid, or wherein said needles are hollow and have a closed tip, wherein said needles having an average tip diameter less than about 10 μm and an average length greater than about 10 μm or greater than about 20 μm wherein said electrically conductive needles are electrically coupled to one or more electrical leads.

### EMBODIMENT 2

The needle electrode of embodiment 1, wherein said needles are solid.

### EMBODIMENT 3

The needle electrode of embodiment 1, wherein said needles are hollow and have a closed tip.

### EMBODIMENT 4

The needle electrode according to any one of embodiments 1-3, wherein said electrode comprises at least about 10 needles, or at least about 15 needles, or at least about 20 needles, or at least about 25 needles, or at least about 30 needles, or at least about 40 needles, or at least about 50 needles, or at least about 100 needles, or at least about 200 needles, or at least about 300 needles, or at least about 400 needles, or at least about 500 needles, or at least about 600 needles, or at least about 700 needles, or at least about 800 needles, or at least about 900 needles, or at least about 1000 needles.

### EMBODIMENT 5

The needle electrode according to any one of embodiments 1-4, wherein said needles are of sufficient length to penetrate at least 60%, or at least 70%, or at least 80%, or at least 90%, or at least 100% through the stratum corneum of the skin when the electrode is attached to the surface of a human over the spinal cord.

### EMBODIMENT 6

The needle electrode according to any one of embodiments 1-5, wherein the needles are of a length that does not substantially penetrate subcutaneous tissue below the stratum corneum.

### EMBODIMENT 7

The needle electrode according to any one of embodiments 1-5, wherein the average length of said ranges from about 1 μm up to about 100 μm, or from about 1 μm up to about 80 μm, or from about 1 μm up to about 50 μm, or from about 1 μm up to about 30 μm, or from about 1 μm up to about 20 μm, or is at least about 30 μm, or at least about 40 μm, or at least about 50 μm, or at least about 60 μm, or at least about 70 μm.

### EMBODIMENT 8

The needle electrode according to any one of embodiments 1-7, the average length of said needles is less than about 200 μm, or less than about 150 μm, or less than about 100 μm.

### EMBODIMENT 9

The needle electrode according to any one of embodiments 1-5, wherein the average length of said needles ranges from about 40 to about 60 μm.

### EMBODIMENT 10

The needle electrode according to any one of embodiments 1-5, wherein the average length of said needles is about 50 μm.

### EMBODIMENT 11

The needle electrode according to any one of embodiments 1-10, wherein the tip of said needles ranges in diameter (or maximum cross-sectional dimension) from about 0.1 μm up to about 10 μm, or from about 0.5 μm up to about 6 μm, or from about 1 μm up to about 4 μm.

### EMBODIMENT 12

The needle electrode according to any one of embodiments 1-11, wherein the average separation between two adjacent needles ranges from about 0.01 mm up to about 1 mm, or about 0.05 mm up to about 0.5 mm, or about 0.1 mm up to about 0.4 mm, or up to about 0.3 mm, or up to about 0.2 mm.

### EMBODIMENT 13

The needle electrode according to any one of embodiments 1-12, wherein the average separation between two adjacent needles ranges from about 0.15 mm up to about 0.25 mm.

### EMBODIMENT 14

The needle electrode according to any one of embodiments 1-13, wherein said needles are disposed in an area of about 1 cm² or less, or about 0.8 cm² or less, or about 0.6 cm² or less, or about 0.5 cm² or less, or about 0.4 cm² or less, or about 0.3 cm² or less, or about 0.2 cm² or less, or about 0.1 cm² or less.

### EMBODIMENT 15

The needle electrode according to any one of embodiments 1-14, wherein said needles are disposed in an area of about 2 mm², or about 3 mm², or about 4 mm², or about 5 mm², or about 6 mm², or about 7 mm² or about 8 mm², or about 9 mm², or about 10 mm².

### EMBODIMENT 16

The needle electrode according to any one of embodiments 1-14, wherein said electrode comprises about 20×about 20 needles in an area about 4×4 mm.

### EMBODIMENT 17

The needle electrode according to any one of embodiments 1-16, wherein the needles including said electrode are substantially uniformly distributed.

### EMBODIMENT 18

The needle electrode according to any one of embodiments 1-17, wherein the needles including said needle electrode are unevenly distributed.

### EMBODIMENT 19

The needle electrode of embodiment 18, wherein the spacing of needles including said electrode is denser at the periphery of said electrode and less dense at the center of said electrode.

### EMBODIMENT 20

The needle electrode of embodiment 18, wherein the spacing of needles including said electrode is denser in the center of the electrode and less dense at the periphery of said electrode.

### EMBODIMENT 21

The needle electrode of embodiment 18, wherein the spacing of needles including said electrode increases in density from one edge of the electrode to the opposite edge of the electrode.

### EMBODIMENT 22

The needle electrode according to any one of embodiments 1-21, wherein said electrode at 10 kHz stimulation frequency has an electrode skin impedance less than ½ the electrode skin impedance of a flat silver chloride (AgCl) electrode having the same projected area.

### EMBODIMENT 23

The needle electrode according to any one of embodiments 1-22, wherein a micro-needle array with 20×20 needles in a 4×4 mm² electrode unit provides an electrode-skin interface impedance at 10 kHz stimulation frequency, of less than about 0.5 Ω/cm2, or less than about 0.249 Ω/cm2.

### EMBODIMENT 24

The needle electrode according to any one of embodiments 1-23, wherein said needles are fabricated from a material selected from the group consisting of platinum, titanium, chromium, iridium, tungsten, gold, carbon nanotubes, stainless steel, silver, silver chloride, indium tin oxide (ITO), conductive polymers (Polypyrrole (Ppy) or poly-3,4-ethylenedioxythiophene (PEDOT)).

### EMBODIMENT 25

The needle electrode according to any one of embodiments 1-23, wherein said needles are fabricated from a material selected from the group consisting of platinum, titanium, chromium, iridium, tungsten, gold, stainless steel, silver, tin, indium, indium tin oxide, oxides thereof, nitrides thereof, and alloys thereof.

### EMBODIMENT 26

The needle electrode according to any one of embodiments 1-25, wherein different needles including said electrode can be independently stimulated.

### EMBODIMENT 27

The needle electrode according to any one of embodiments 1-25, wherein said needles are electrically coupled to each other and can be stimulated as a group.

### EMBODIMENT 28

The needle electrode according to any one of embodiments 1-27, wherein said electrode array when attached to the skin surface over the spinal cord can stimulate the spinal cord without the use of a conductive gel or cream disposed between the electrode and the skin.

### EMBODIMENT 29

The needle electrode according to any one of embodiments 1-28, wherein said electrode, when applied to the skin over a region of the spinal cord can conduct a signal having frequency and amplitude sufficient to stimulate the spinal cord without degradation of the electrode.

### EMBODIMENT 30

The needle electrode according to any one of embodiments 1-29, wherein said needle electrode has hollow grids between the needles including said electrode.

### EMBODIMENT 31

The needle electrode according to any one of embodiments 1-30, wherein said needle electrode is attached to a conventional transcutaneous electrical stimulation electrode.

### EMBODIMENT 32

The needle electrode according to any one of embodiments 1-31, wherein said electrode is disposed on a flexible backing.

### EMBODIMENT 33

The needle electrode of embodiment 32, wherein said flexible backing comprises a polymer.

### EMBODIMENT 34

The needle electrode of embodiment 33, wherein said flexible backing comprise a polymer selected from the group consisting of polyimide, parylene, PVC, polyethylene, PEEK, polycarbonate, Ultem PEI, polysulfone, polypropylene, and polyurethane.

### EMBODIMENT 35

The needle electrode according to any one of embodiments 32-34, wherein said backing comprises a plurality of holes that provide heat and moisture dissipation.

### EMBODIMENT 36

The needle electrode according to any one of embodiments 32-35, wherein said backing comprises an adhesive for attachment to the skin surface.

### EMBODIMENT 37

The needle electrode according to any one of embodiments 32-35, wherein said electrode and/or said backing comprises one or more sensors.

### EMBODIMENT 38

The needle electrode of embodiment 37, wherein said electrode and/or said backing comprises a temperature sensor.

### EMBODIMENT 39

The needle array according to any one of embodiments 37-38, wherein said electrode and/or said backing comprises a flex sensor and/or pressure sensor to monitor change in position and pressure forces to the skin and/or electrode.

### EMBODIMENT 40

The needle array according to any one of embodiments 37-38, wherein said electrode and/or said backing comprises a photonic sensor to monitor blood flow.

### EMBODIMENT 41

An electrode array including a plurality of needle electrodes according to any one of embodiments 1-40.

### EMBODIMENT 42

The electrode array of embodiment 41, wherein said electrode array comprises at least three needle electrodes, or at least four needle electrodes, or at least 5 needle electrodes, or at least 6 needle electrodes, or at least 7 needle electrodes, or at least 8 needle electrodes, or at least 9 needle electrodes, or at least 10 needle electrodes, or at least 15 needle electrodes, or at least 20 needle electrodes, or at least 25 needle electrodes, or at least 30 needle electrodes, or at least 35 needle electrodes, or at least 40 needle electrodes, or at least 45 needle electrodes, or at least 50 needle electrodes, or at least 75 needle electrodes, or at least 100 needle electrodes.

### EMBODIMENT 43

The electrode array according to any one of embodiments 41-42, wherein said needle electrodes are disposed on a common backing.

### EMBODIMENT 44

The electrode array of embodiment 43, wherein said common backing is a flexible backing.

### EMBODIMENT 45

The electrode array of embodiment 44, wherein said flexible backing comprises a polymer.

### EMBODIMENT 46

The electrode array of embodiment 45, wherein said flexible backing comprise a polymer selected from the group consisting of polyimide, parylene, PVC, polyethylene, PEEK, polycarbonate, Ultem PEI, polysulfone, polypropylene, and polyurethane.

### EMBODIMENT 47

The electrode array according to any one of embodiments 43-46, wherein said common backing comprises a plurality of holes that provide heat and moisture dissipation.

### EMBODIMENT 48

The electrode array according to any one of embodiments 43-47, wherein said common backing comprises an adhesive for attachment to the skin surface.

### EMBODIMENT 49

The electrode array according to any one of embodiments 41-42, wherein different needle electrodes including said plurality of needle electrodes are disposed on different backings.

### EMBODIMENT 50

The electrode array according to any one of embodiments 41-49, wherein different needle electrodes including said plurality of needle electrodes are coupled to different electrical leads such that different electrical signals can be applied to different needle electrodes.

### EMBODIMENT 51

The electrode array according to any one of embodiments 41-50, wherein one or more electrodes including said array are configured to deliver a transcutaneous stimulation signal and one or more electrodes including said array are configured to provide a ground or return.

### EMBODIMENT 52

The electrode array according to any one of embodiments 41-51, wherein one or more needle electrodes is configured to record an electrical potential.

### EMBODIMENT 53

The electrode array according to any one of embodiments 41-52, wherein the electrode array and/or assembled package incorporates one or more sensors.

### EMBODIMENT 54

The electrode array of embodiment 53, wherein said sensor(s) are selected from the group consisting of a temperature sensor, a flex sensor and/or pressure sensor, and a photonic sensor that measures blood flow.

### EMBODIMENT 55

The electrode array according to any one of embodiments 41-54, wherein the electrode array is wireless or contains wireless capabilities.

### EMBODIMENT 56

A system for transcutaneous simulation of the spinal cord and/or brain, said system including: a needle electrode according to any one of embodiments 1-40 or an electrode array according to any one of embodiments 41-55; and an electrical stimulator configured to deliver transcutaneous stimulation to the brain or spinal cord through one or more electrodes including said electrode array or electrode array assembly.

### EMBODIMENT 57

The system of embodiment 56, wherein said system is configured to provide transcutaneous stimulation signal at a frequency ranging from about 0.3 Hz, or from about 1 Hz, or from about 3 Hz, or from about 5 Hz, or from about 10 Hz up to about 50 kHz, or up to about 30 kHz, or up to about 20 kHz, or up to about 10 kHz, or up to about 1,000 Hz, or up to about 500 Hz, or up to about 100 Hz, or up to about 80 Hz, or up to about 40 Hz, or from about 3 Hz or from about 5 Hz up to about 80 Hz, or from about 5 Hz up to about 30 Hz, or up to about 40 Hz, or up to about 50 Hz.

### EMBODIMENT 58

The system according to any one of embodiments 56-57, wherein said system is configured to provide transcutaneous stimulation signal at an amplitude ranging from 10 mA to about 500 mA or up to about 300 mA, or up to about 150 mA, or from about 20 mA up to about 50 mA or up to about 100 mA, or from about 20 mA or from about 30 mA, or from about 40 mA up to about 50 mA, or up to about 60 mA, or up to about 70 mA, or up to about 80 mA.

### EMBODIMENT 59

The system according to any one of embodiments 56-58, wherein system is configured to provide a transcutaneous stimulation signal pulse width that ranges from about 100 μs up to about 5000 μs, or from about 100 μs up to about 1000 μs, or from about 150 μs up to about 600 μs, or from about 200 μs up to about 500 μs, or from about 200 μs up to about 450 μs.

### EMBODIMENT 60

The system according to any one of embodiments 56-59, wherein said system is configured to deliver said transcutaneous stimulation signal superimposed on a high frequency carrier signal.

### EMBODIMENT 61

The system of embodiment 60, wherein said high frequency carrier signal ranges from about 3 kHz, or about 5 kHz, or about 8 kHz up to about 100 kHz, or up to about 80 kHz, or up to about 50 kHz, or up to about 40 kHz, or up to about 30 kHz, or up to about 20 kHz, or up to about 15 kHz.

### EMBODIMENT 62

The system of embodiment 60, wherein said high frequency carrier signal is about 10 kHz.

### EMBODIMENT 63

The system according to any one of embodiments 60-62, wherein said carrier frequency amplitude ranges from about 30 mA, or about 40 mA, or about 50 mA, or about 60 mA, or about 70 mA, or about 80 mA up to about 500 mA, or up to about 400 mA, or up to about 300 mA, or up to about 200 mA, or up to about 150 mA.

### EMBODIMENT 64

The system according to any one of embodiments 56-63, wherein said system is configured to provide transcutaneous stimulation at a frequency and amplitude sufficient to stimulate and/or to improve postural and/or locomotor activity and/or postural or locomotor strength.

### EMBODIMENT 65

The system according to any one of embodiments 56-63, wherein said system is configured to provide transcutaneous stimulation at a frequency and amplitude sufficient to stimulate and/or to improve reaching and/or grasping and/or fine motor control of a hand.

### EMBODIMENT 66

The system according to any one of embodiments 56-63, wherein said system is configured to provide transcutaneous stimulation at a frequency and amplitude sufficient to stimulate voluntary voiding of the bladder and/or bowel, and/or return of sexual function, and/or autonomic control of cardiovascular function, and/or body temperature, control of digestive functions, control of kidney functions, chewing, swallowing, drinking, talking, or breathing.

### EMBODIMENT 67

The system according to any one of embodiments 56-63, wherein said system is configured to provide transcutaneous stimulation at a frequency and amplitude sufficient to stimulate voluntary voiding of the bladder and/or bowel, and/or return of sexual function, and/or autonomic control of cardiovascular function, and/or body temperature, control of digestive functions, control of kidney functions, chewing, swallowing, drinking, talking, or breathing.

### EMBODIMENT 68

The system according to any one of embodiments 56-67, wherein the electrode and/or backing comprises a temperature sensor.

### EMBODIMENT 69

The system of embodiment 68, wherein said system is configured to turn off a signal to the electrode when the temperature reaches a critical value.

### EMBODIMENT 70

The system according to any one of embodiments 56-38, wherein said electrode and/or said backing comprises a flex sensor and/or pressure sensor to monitor change in position and pressure forces to the skin and/or electrode.

### EMBODIMENT 71

The system of embodiment 70, wherein said system is configured to alter or turn off stimulation in response to changes in position and/or pressure forces.

### EMBODIMENT 72

The system according to any one of embodiments 56-71, wherein said electrode and/or said backing comprises a photonic sensor to monitor blood flow.

### EMBODIMENT 73

A method of stimulating or improving postural and/or locomotor activity and/or postural or locomotor strength, and/or reaching or grasping, and/or fine motor control of a hand, and/or enabling one or more functions selected from the group consisting of voluntary voiding of the bladder and/or bowel, return of sexual function, autonomic control of cardiovascular function, and body temperature control, control of digestive functions, control of kidney functions, chewing, swallowing, drinking, talking, or breathing in a normal subject or a subject having a neurologically derived paralysis said method including neuromodulating the spinal cord, brainstem, or brain of said subject or a region thereof by administering transcutaneous stimulation to the spinal cord or a region thereof using an electrical stimulator electrically coupled to a needle electrode according to any one of embodiments 1-40 or an electrode array according to any one of embodiments 41-55, wherein said needle electrode or at least a part of said electrode array is disposed on the skin surface over the spinal cord or a region thereof.

### EMBODIMENT 74

The method of embodiment 73, wherein said transcutaneous stimulation is at a frequency ranging from about 0.5 Hz or from about 3 Hz, or from about 5 Hz, or from about 10 Hz up to about 50 kHz, or up to about 30 kHz, or up to about 20 kHz, or up to about 10 kHz, or up to about 1,000 Hz, or up to about 500 Hz, or up to about 100 Hz, or up to about 80 Hz, or up to about 40 Hz, or from about 3 Hz or from about 5 Hz up to about 80 Hz, or from about 5 Hz up to about 30 Hz, or up to about 40 Hz, or up to about 50 Hz.

### EMBODIMENT 75

The method according to any one of embodiments 73-74, wherein said transcutaneous stimulation is at an amplitude ranging from 10 mA to about 500 mA, or up to about 300 mA, or up to about 150 mA, or from about 20 mA to about 300 mA, or up to about 50 mA or up to about 100 mA, or from about 20 mA or from about 30 mA, or from about 40 mA to about 50 mA, or to about 60 mA, or to about 70 mA or to about 80 mA.

### EMBODIMENT 76

The method according to any one of embodiments 73-75, wherein said transcutaneous stimulation pulse width ranges from about 100 μs up to about 1000 μs, or from about 150 μs up to about 600 μs, or from about 200 μs up to about 500 μs, or from about 200 μs to about 450 μs.

### EMBODIMENT 77

The method according to any one of embodiments 73-76, wherein said transcutaneous stimulation is superimposed on a high frequency carrier signal.

### EMBODIMENT 78

The method of embodiment 77, wherein said high frequency carrier signal ranges from 3 kHz, or about 5 kHz, or about 8 kHz up to about 100 kHz, or up to about 80 kHz, or up to about 50 kHz, or up to about 40 kHz, or up to about 30 kHz, or up to about 20 kHz, or up to about 15 kHz.

### EMBODIMENT 79

The method of embodiment 77, wherein said high frequency carrier signal is about 10 kHz.

### EMBODIMENT 80

The method according to any one of embodiments 77-79, wherein said carrier frequency amplitude ranges from about 30 mA, or about 40 mA, or about 50 mA, or about 60 mA, or about 70 mA, or about 80 mA up to about 500 mA, or up to about 300 mA, or up to about 200 mA, or up to about 150 mA.

### EMBODIMENT 81

The method according to any one of embodiments 73-80, wherein said transcutaneous stimulation is at a frequency and amplitude sufficient to stimulate and/or to improve postural and/or locomotor activity and/or postural or locomotor strength.

### EMBODIMENT 82

The method according to any one of embodiments 73-80, wherein said transcutaneous stimulation is at a frequency and amplitude sufficient to stimulate and/or improve reaching and/or grasping and/or fine motor control of a hand.

### EMBODIMENT 83

The method according to any one of embodiments 73-80, wherein said transcutaneous stimulation is at a frequency and amplitude sufficient to stimulate voluntary voiding of the bladder and/or bowel, and/or return of sexual function, and/or autonomic control of cardiovascular function, and/or body temperature, control of digestive functions, control of kidney functions, chewing, swallowing, drinking, talking, or breathing.

### EMBODIMENT 84

The method according to any one of embodiments 73-83, wherein said transcutaneous stimulation is applied on the skin surface over the cervical spine or a region thereof and/or over the thoracic spine or a region thereof, and/or over the lumbosacral spine or a region thereof.

### EMBODIMENT 85

The method according to any one of embodiments 73-83, wherein said transcutaneous stimulation is applied on the skin surface over a region of the spinal cord that controls the lower limbs upper limbs to stimulate or improve postural and/or locomotor activity and/or postural or locomotor strength.

### EMBODIMENT 86

The method of embodiment 85, wherein said locomotor activity comprises standing and/or stepping.

### EMBODIMENT 87

The method of embodiment 85, wherein said locomotor activity comprises sitting down or laying down.

### EMBODIMENT 88

The method of embodiment 85, wherein said movement comprises stabilizing sitting or standing posture.

### EMBODIMENT 89

The method according to any one of embodiments 73-83, wherein said transcutaneous stimulation is applied on the skin surface over a region of the spinal cord that controls the upper limbs to improve reaching and/or grasping and/or to improve improving motor control and/or strength in a hand and/or upper limb of a subject with a neuromotor disorder affecting motor control of the hand and/or upper limb.

### EMBODIMENT 90

The method according to any one of embodiments 85-88, wherein said method comprises subjecting said subject to physical training that exposes said subject to relevant postural and locomotor or motor proprioceptive signals.

### EMBODIMENT 91

The method of embodiment 90, wherein the wherein the combination of said stimulation and physical training modulates in real time the electrophysiological properties of spinal circuits in said subject so they are activated by proprioceptive information derived from the region of the subject where said previously stated functions are facilitated.

### EMBODIMENT 92

The method according to any one of embodiments 90-91, wherein said physical training comprises inducing a load bearing positional change in the region of the subject where locomotor activity is to be facilitated.

### EMBODIMENT 93

The method according to embodiment 92, wherein the load bearing positional change in said subject comprises standing.

### EMBODIMENT 94

The method according to embodiment 92, wherein the load bearing positional change in said subject comprises stepping.

### EMBODIMENT 95

The method according to embodiment 92, wherein the load bearing positional change in said subject comprises reaching.

### EMBODIMENT 96

The method according to embodiment 92, wherein the load bearing positional change in said subject comprises grasping.

### EMBODIMENT 97

The method according to any one of embodiments 85-96, wherein said physical training comprises robotically guided training.

### EMBODIMENT 98

The method according to any one of embodiments 85-97, wherein said physical training comprises hand contraction and/or upper limb movements against a resistance.

### EMBODIMENT 99

The method according to any one of embodiments 85-97, wherein said physical training comprises tracing a displayed pattern by hand manipulation of a hand controller.

### EMBODIMENT 100

The method according to any one of embodiments 73-99, wherein said transcutaneous stimulation is applied over a region of the spinal cord that controls the bladder and/or bowel.

### EMBODIMENT 101

The method according to any one of embodiments 73-100, wherein one or more needle electrodes are stimulated in a monopolar configuration.

### EMBODIMENT 102

The method according to any one of embodiments 73-100, wherein one or more needle electrodes are stimulated in a monophasic configuration.

### EMBODIMENT 103

The method according to any one of the embodiments 73-100, wherein one or more needle electrodes are stimulated in a biphasic configuration.

### EMBODIMENT 104

The method according to any one of embodiments 73-100, wherein one or more needle electrodes are stimulated in a bipolar configuration.

### EMBODIMENT 105

The method according to any one of embodiments 73-104, wherein said stimulation comprises tonic stimulation.

### EMBODIMENT 106

The method according to any one of embodiments 73-105, wherein said stimulation comprises simultaneous or sequential stimulation of different spinal cord regions.

### EMBODIMENT 107

The method according to any one of embodiments 73-106, wherein the stimulation pattern is under control of the subject.

### EMBODIMENT 108

The method according to any one of embodiments 73-107, wherein one or more needle electrodes is used to record an electrical potential.

### EMBODIMENT 109

The electrode array according to any one of embodiments 73-108, where said method comprises monitoring a temperature sensor and turning off stimulation if the temperature exceeds a critical value.

### EMBODIMENT 110

The method according to any one of embodiments 73-109, wherein said subject is administered at least one neuromodulatory drug.

### EMBODIMENT 111

The method according to any one of embodiments 73-109, wherein said subject is administered at least one monoaminergic agonist.

### EMBODIMENT 112

The method of embodiment 111, wherein said at least one monoaminergic agonist comprises an agent selected from the group consisting of a serotonergic drug, a dopaminergic drug, a noradrenergic drug, a GABAergic drug, and a glycinergic drug.

### EMBODIMENT 113

The method of embodiment 112, wherein said agent is selected from the group consisting of 8-hydroxy-2-(di-n-propylamino)tetralin (8-OH-DPAT), 4-(benzodioxan-5-yl)1-(indan-2-yl)piperazine (S15535), N-{2-[4-(2-methoxyphenyl)-1-piperazinyl]ethyl}-N-(2-pyridinyl)cyclo-hexanecarboxamide (WAY 100.635), Quipazine, Ketanserin, 4-amino-(6-chloro-2-pyridyl)-1 piperidine hydrochloride (SR 57227A), Ondanesetron, Buspirone, Methoxamine, Prazosin, Clonidine, Yohimbine, 6-chloro-1-phenyl-2,3,4,5-tetrahydro-1H-3-benzazepine-7,8-diol (SKF-81297), 7-chloro-3-methyl-1-phenyl-1,2,4,5-tetrahydro-3-benzazepin-8-ol (SCH-23390), Quinpirole, and Eticlopride.

### EMBODIMENT 114

The method of embodiment 112, wherein said monoaminergic agonist is buspirone.

### EMBODIMENT 115

The method of embodiment 110, wherein said neuromodulatory drug is a molecule that activates (e.g., selectively activates) an α2c adrenergic receptor subtype and/or that blocks (e.g., selectively blocks) blocking an α2a adrenergic receptor subtype.

### EMBODIMENT 116

The method of embodiment 115, wherein said molecule that activates an α2c adrenergic receptor subtype is 2-[(4,5-Dihydro-1H-imidazol-2-yl)methyl]-2,3-dihydro-1-methyl-1H-isoindole (BRL-44408).

### EMBODIMENT 117

The method of embodiment of 115, said molecule that activates an α2c adrenergic receptor subtype is (R)-3-nitrobiphenyline and/or a compound according to the formula:

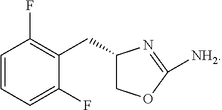 [SMILES: NC1=N[C@@H](Cc2c(F)cccc2F)CO1]

### EMBODIMENT 118

The method of embodiment 115, wherein said agonist agonist is Clonidine.

### EMBODIMENT 119

The method of embodiment 115, wherein said neuromodulatory drug further comprises a 5-HT1 and/or a 5-HT7 serotonergic agonist.

### EMBODIMENT 120

The method according to any one of embodiments 73-119, wherein said subject is a human.

### EMBODIMENT 121

The method according to any one of embodiments 73-119, wherein said subject has a spinal cord injury.

### EMBODIMENT 122

The method of embodiment 121, wherein said spinal cord injury is clinically classified as motor complete.

### EMBODIMENT 123

The method of embodiment 121, wherein said spinal cord injury is clinically classified as motor incomplete.

### EMBODIMENT 124

The method according to any one of embodiments 73-120, wherein said subject has an ischemic brain injury.

### EMBODIMENT 125

The method of embodiment 124, wherein said ischemic brain injury is brain injury from stroke or acute trauma.

### EMBODIMENT 126

The method according to any one of embodiments 73-120, wherein said subject has a neurodegenerative pathology.

### EMBODIMENT 127

The method of embodiment 126, wherein said neurodegenerative pathology is associated with a condition selected from the group consisting of stroke, Parkinson's disease, Huntington's disease, Alzheimer's disease, amyotrophic lateral sclerosis (ALS), primary lateral sclerosis (PLS), dystonia, and cerebral palsy.

### EMBODIMENT 128

A method of fabricating a needle electrode according to any one of embodiments 1-40, said method including: 3-D printing and/or laser cutting a 3-D printable or laser cuttable material into the shape of said needle electrode to form a needle electrode model; and depositing a metal on said form to provide said needle electrode.

### EMBODIMENT 129

A method of fabricating a needle electrode according to any one of embodiments 1-40, said method including: 3-D printing and/or laser cutting a 3-D printable or laser cuttable material to form a mold of the needle array; fabricating said needle array by hot embossing said mold; and depositing a metal on the hot-embossed structure to provide said needle electrode.

### EMBODIMENT 130

A method of fabricating a needle electrode according to any one of embodiments 1-40, said method including: metal stamping said needle array.

### EMBODIMENT 131

A method of fabricating a needle electrode according to any one of embodiments 1-40, said method including: electrical discharge machining said needle array.

### EMBODIMENT 132

A method of fabricating a needle electrode according to any one of embodiments 1-40, said method including: providing a substrate with tapered holes; depositing a material onto the substrate with etched tunnel structures that terminate on the tapering surface of a hole; depositing an electrode substrate into the tunnel structures and to form a needle electrode substrate; and depositing a biocompatible metal on said needle electrode substrate to produce needle electrode.

### EMBODIMENT 133

The method according to any one of embodiments 128-132, wherein said biocompatible metal comprises a material selected from the group consisting of platinum, titanium, chromium, iridium, tungsten, gold, carbon-nanotubes, stainless steel, silver, silver chloride, indium tin oxide (ITO), and a conductive polymer (e.g., Polypyrrole (Ppy) or poly-3,4-ethylenedioxythiophene (PEDOT)).

### Definitions

The term “motor complete” when used with respect to a spinal cord injury indicates that there is no motor function below the lesion, (e.g., no movement can be voluntarily induced in muscles innervated by spinal segments below the spinal lesion.

As used herein “electrical stimulation” or “stimulation” means application of an electrical signal that may be either excitatory or inhibitory to a muscle, nerve, nerve cell body, nerve root, a neuron or neurons, a network of nerve fibers, the spinal cord, brainstem, and/or brain. It will be understood that an electrical signal may be applied to one or more electrodes with one or more return electrodes.

The term “monopolar stimulation” refers to stimulation between a local electrode and a common distant return electrode.

The term “bipolar stimulation” refers to stimulation between two closely spaced electrodes.

The term “transcutaneous stimulation” or “transcutaneous electrical stimulation” or “cutaneous electrical stimulation” refers to electrical stimulation applied to the skin, and, as typically used herein refers to electrical stimulation applied to the skin in order to effect stimulation of the spinal cord or a region thereof. The term “transcutaneous electrical spinal cord stimulation” may also be referred to as “tSCS”.

The term “autonomic function” refers to functions controlled by the central nervous system that are controlled largely below the level of consciousness, and typically involve visceral functions. Illustrative autonomic functions include, but are not limited to control of bowel, bladder, and body temperature.

The term “sexual function” refers to the ability to sustain a penile erection, have an orgasm (male or female), generate viable sperm, and/or undergo an observable physiological change associated with sexual arousal.

The term “co-administering”, “concurrent administration”, “administering in conjunction with” or “administering in combination” when used, for example with respect to transcutaneous electrical stimulation, epidural electrical stimulation, and pharmaceutical administration, refers to administration of the transcutaneous electrical stimulation and/or epidural electrical stimulation and/or pharmaceutical such that various modalities can simultaneously achieve a physiological effect on the subject. The administered modalities need not be administered together, either temporally or at the same site. In some embodiments, the various “treatment” modalities are administered at different times. In some embodiments, administration of one can precede administration of the other (e.g., drug before electrical stimulation or vice versa). Simultaneous physiological effect need not necessarily require presence of drug and the electrical stimulation at the same time or the presence of both stimulation modalities at the same time. In some embodiments, all the modalities are administered essentially simultaneously.

### BRIEF DESCRIPTION OF THE DRAWINGS

FIG. 1 illustrates one embodiment of a needle electrode design that can reduce the electrode-skin interface impedance.

FIG. 2, illustrates the difference between the needle electrode array described herein and traditional TENS electrodes (and conventional one—The corresponding equivalent circuitry is shown respectively. Our proposed surface of the electrode will be designed to protrude the external surface of the Stratum Corneum (SC) (outer skin layer which consists of dead cells) but not penetrate the SG (which consists of live skin cells) or Dermis layer (where peripheral nerves and micro blood vessels exist). Thus the overall larger electrode surface area plus the closer proximity of the current source to the SC and dermis reduces the impedance significantly.

FIG. 3 illustrates the results of an electrode-skin interface impedance measurement. Blue trace: conventional AgCl electrode. Red trace: the needle electrode

FIG. 4 illustrates edge effect than simulation results. Left column: conventional transcutaneous electrical stimulation electrodes (A) voltage distribution (B) electrical field (C) current density. Right column: (D) voltage distribution (E) electrical field (F) current density. Note that the units in both columns are equalized.

FIG. 5 illustrates Fabrication methods (A) Array is built by 3D printing or laser cutting, and followed with metal encapsulation. (B) A mold is built by 3D printing or laser cutting, and then a conventional hot-embossing process is utilized to fabricate the array. Finally, metal encapsulation is applied onto the array. (C) A substrate with cone-shape holes, then a material was deposited onto the substrate with etched tunnel structures right on the cone-shape holes. Afterward, another material which is used as electrode substrate is deposited into the tunnel structure. Deposition/encapsulation of biocompatible metal is then applied onto the released electrode for low impedance contact.

FIG. 6 illustrates assembly and package of needle electrode. Multiple needle electrode units can form a flexible array when being attached onto a flexible handle/adhesion layer.

FIG. 7 illustrates one embodiment of an electrode design.

### DETAILED DESCRIPTION

In various embodiments a novel needle electrode is provided that is suitable for transcutaneous electrical stimulation of the spinal cord. The needle electrodes described herein are well suited for transcutaneous electrical stimulation with low impedance and conformal electrical field distribution.

Previous transcutaneous electrode face a number of difficulties in use including, for example, 1) high electrode-skin interface impedance which, particular under high use can produce skin complications (e.g., irritation and burning); 2) problems with edge effects that reduce overall effectiveness; and 3) are not well suited to provide conformal attachment to the skin surface particularly over extended periods of time (e.g., days to weeks, to months).

A high electrode impedance along with high stimulation current results in the requirement of high compliance voltage. Also, a high stimulation current running with high voltage means high electrical power dissipation through skin, which causes damage including burning and irritation. Methods of reducing the impedance in the commercially available transcutaneous electrodes include surface modification of the electrodes and use of a conductive gel or cream to coat the skin and provide lower impedance. However, the use of conductive gels or creams cause allergic reaction of the skin, and the skin impedance typically increases gradually over time as the gel hardens and/or the cream dries.

In certain embodiments transcutaneous electrical stimulation can uses high frequency modulated with a low frequency to achieve painless and effective stimulation [1]. However, analytical and numerical studies as well as experimental measurements indicate that the current density on a metal disk is spatially non-uniform with very high current density at the edges and much lower current density in the center [2-4]. The non-uniform current density distribution may affect the propensity for stimulation to cause either tissue and/or electrode damage. This edge effect with non-uniform current density causes many problems in applications where current is injected into tissue using metal electrodes. The heat source Q (W/m³) can be calculated by

$$Q = \frac{{\vert J \vert}^{2}}{σ}$$

where J and σ are the current density (A/m²) and electrical conductivity [5]. Because heating increases with power density during RF ablation, peak temperatures occur at the electrode edge at the junction between the electrode and the tissue [6].

To date, most transcutaneous electrical stimulation electrodes consist of a sticky electrical conductive layer on the electrode surface. The conductive layer also works as to attach the electrode to the skin surface. However, the use of such a gel can create an itchy sensation, irritability and general discomfort, particularly under long-time usage. Where a stick gel is omitted, additional methods of attaching the electrode are typically required to enhance the contact between electrodes and skin [7].

The needle electrodes described herein overcome these and other problems. In various embodiments needle electrodes contemplated herein comprise a plurality of electrically conductive solid microprojections (or where the needles are hollow, they are closed at the tip), where the needles (microprojections) have a tip dimension/diameter small enough to facilitate penetration of the stratum corneum on the skin (e.g., less than about 10 μm), where the needles have a length greater than about 20 μm and where the electrically conductive solid needles are electrically coupled to one or more electrical leads.

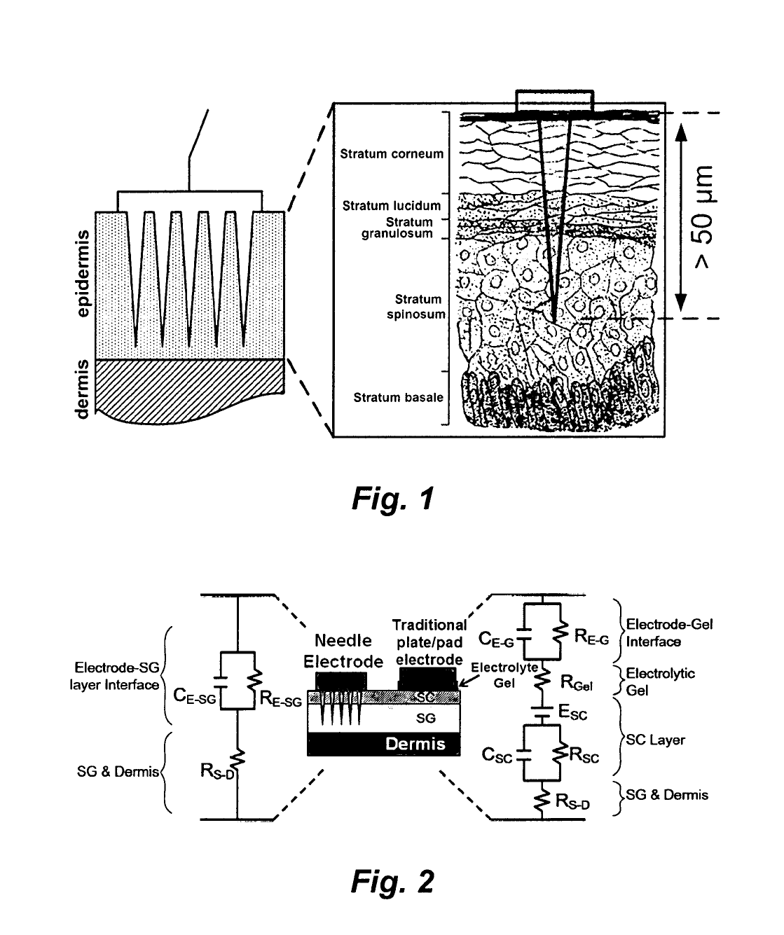

One illustrative, but non-limiting needle electrode is shown in FIG. 1. As illustrated in this figure needles with tip size of several μm or smaller, and a shaft length of 50 μm or more were used in these transcutaneous electrical stimulation electrodes. In one embodiment, a single electrode unit, consisting of 5×5 to 30×30 needles, is about one centimeter in diameter. Multiple electrode units can be further combined into an electrode array, e.g., when larger electrode areas are needed (for example, for the return/ground electrodes). The needle electrodes described herein can provide low impedance transcutaneous stimulation without using a conductive gel or cream.

As illustrated in FIG. 1, a carefully designed needle geometry enables the impalement of needle tips through the outer skin layer (Stratum Corneum, SC) into deeper skin, but not into the subcutaneous tissue, which contains capillary vessels and peripheral nerve. The outer skin layer consists of dead cells therefore has a high electrical resistance (e.g., it is an electrical insulator). The needles of the needle electrodes described herein are able to penetrate into the live skin cells thereby by bypassing the SC layer, and consequently resulting in overall lower impedance than at the SC layer, as shown in FIG. 2. Since the needle does not reach the subcutaneous tissue, there is no pain or bleeding.

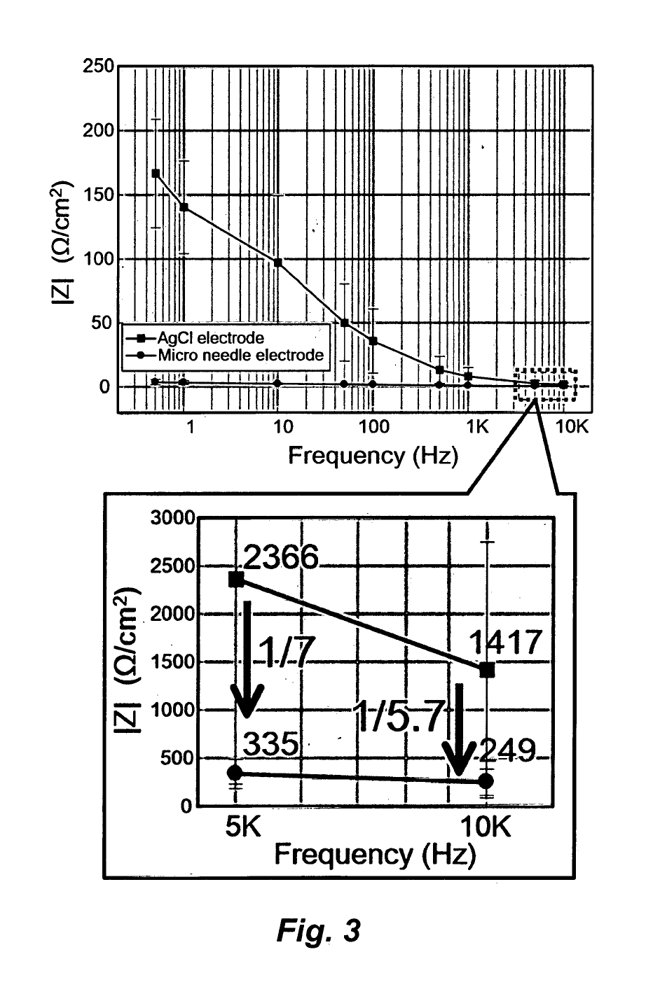

FIG. 3 demonstrates that the needle array with 20×20 needles in a 4×4 mm² electrode unit results in significantly reducing the electrode-skin interface impedance. Specifically at 10 kHz stimulation frequency, impedance of a conventional silver chloride (AgCl) electrode and micro-needle electrode are 1.416 and 0.249 Ω/cm2 respectively. Thus, the needle electrode as a 5.7 times lower impedance. This means that the compliance voltage and in turn the total electrical power is reduced almost 6 times if a micro-needle electrode design is used. At lower stimulation frequencies, the improvement is even greater.

Accordingly, in certain embodiments the electrode is configured such that the electrode at 10 kHz stimulation frequency has an electrode skin impedance less than ½ the electrode skin impedance of a flat silver chloride (AgCl) electrode having the same projected area. For example, in certain embodiments, a micro-needle array with 20×20 needles in a 4×4 mm² electrode unit provides an electrode-skin interface impedance at 10 kHz stimulation frequency, of less than about 0.5 Ω/cm2, or less than about 0.249 Ω/cm2.

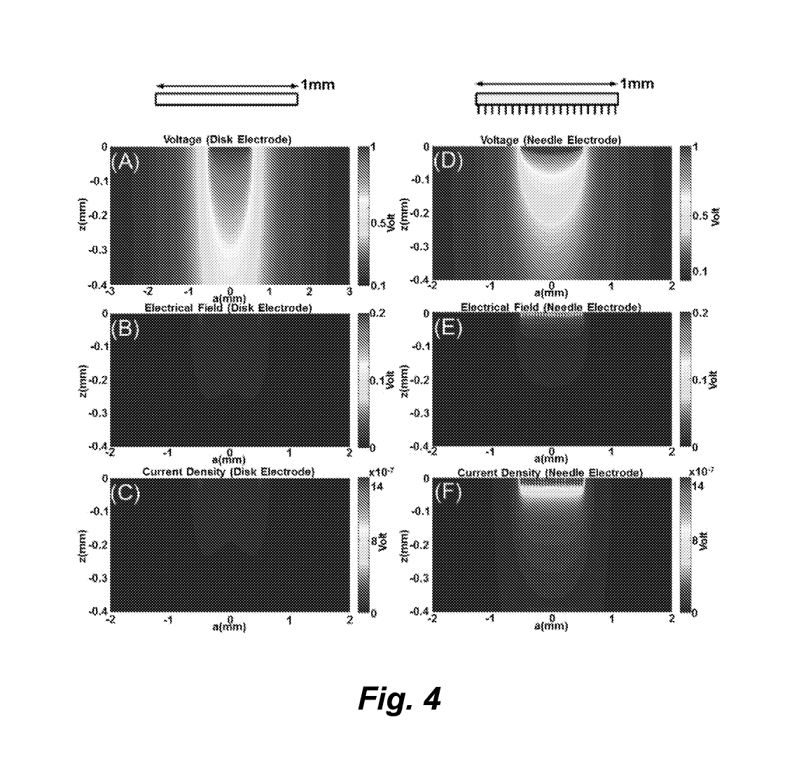

FIG. 4 shows a simulation of the current density induced by the needles electrode. In this simulation, both a planar disk electrode and the needle electrode are 1 mm in diameter, while the separation between two needles on the needle electrode is 0.05 mm. In the simulation, a 1V voltage was applied onto the electrode. The voltage (potential) distribution can be obtained according to [2], as shown in FIG. 4, panels (A) and (D):

$$V ( r , z ) = \frac{2 {V}_{o}}{π} {sin}^{- 1} \{ \frac{2 a}{{[ {( r - a )}^{2} + {z}^{2} ]}^{1 / 2} + {[ {( r + a )}^{2} + {z}^{2} ]}^{1 / 2}} \}$$

where r and a are the radius of region of interest and the electrode, z is the depth into the skin. Then the electrical field and current density can be calculated by

\vec{E}=−\vec{∇}V

\vec{J}=σ\vec{E}

where σ is the conductivity. The results are shown in FIG. 4, panels (B), (E) and (C), (F) for the electrical field and current density of the disk electrode and needle electrode, respectively. As shown in the FIG. 4, since the current will travels through the low resistance path created by the needles that touch the deeper skin layer, a relative uniform current density can be induced. Furthermore, the more needles on the electrode, more uniform electrical field and current density we can achieve.

The needle electrodes contemplated herein are not limited to the embodiments illustrated in the Figures. In certain embodiments the needle electrode comprises needles a plurality of which are of sufficient length to penetrate at least 60%, or at least 70%, or at least 80%, or at least 90%, or at least 100% through the stratum corneum of the skin when the electrode is attached to the surface of a human over the spinal cord. In certain embodiments the needles are of a length that does not substantially penetrate subcutaneous tissue below the stratum corneum. In certain embodiments the average length of said needles ranges from about 1 μm up to about 200 μm, or from about 1 μm up to about 100 μm, or from about 1 μm up to about 80 μm, or from about 1 μm up to about 50 μm, or from about 1 μm up to about 30 μm, or from about 1 μm up to about 20 μm, or is at least about 30 μm, or at least about 40 μm, or at least about 50 μm, or at least about 60 μm, or at least about 70 μm. In certain embodiments the average length of said needles is less than about 200 μm, or less than about 150 μm, or less than about 100 μm. In one illustrative, but non-limiting embodiment, the average length of said needles ranges from about 40 to about 60 μm (e.g., about 50 μm). In certain embodiments the tip of the needles ranges in diameter (or maximum cross-sectional dimension) from about 0.1 μm up to about 10 μm, or from about 0.5 μm up to about 6 μm, or from about 1 μm up to about 4 μm.

In certain embodiments the needles comprising the electrode are substantially conical in shape (e.g., they have an approximately circular cross-section). In certain embodiments the cross-section of the needles is a different regular polygon (e.g., triangular, square, pentagonal, hexagonal, octagonal, etc.). In certain embodiments, the needle cross-section is an irregular polygon (e.g., rectangular, trapezoidal, etc.), oval, or another irregular shape.

In certain embodiments the needle electrode comprises at least 4, or at least 6, or at least 8, or at least about 10 needles, or at least about 15 needles, or at least about 20 needles, or at least about 25 needles, or at least about 30 needles, or at least about 40 needles, or at least about 50 needles, or at least about 100 needles, or at least about 200 needles, or at least about 300 needles, or at least about 400 needles, or at least about 500 needles, or at least about 600 needles, or at least about 700 needles, or at least about 800 needles, or at least about 900 needles, or at least about 1000 needles.

In certain embodiments the average separation between two adjacent needles ranges from about 0.01 mm up to about 1 mm, or about 0.05 mm up to about 0.5 mm, or about 0.1 mm up to about 0.4 mm, or up to about 0.3 mm, or up to about 0.2 mm. In certain embodiments the average separation between two adjacent needles ranges from about 0.15 mm up to about 0.25 mm. In certain embodiments the needles are disposed in an area of about 1 cm² or less, or about 0.8 cm² or less, or about 0.6 cm² or less, or about 0.5 cm² or less, or about 0.4 cm² or less, or about 0.3 cm² or less, or about 0.2 cm² or less, or about 0.1 cm² or less. In certain embodiments the needles are disposed in an area of about 2 mm or about 3 mm, or about 4 mm, or about 5 mm, or about 6 mm, or about 7 mm or about 8 mm, or about 9 mm, or about 10 mm by about 2 mm or about 3 mm, or about 4 mm, or about 5 mm, or about 6 mm, or about 7 mm or about 8 mm, or about 9 mm, or about 10 mm. In one illustrative, but non-limiting embodiment the electrode comprises about 20×about 20 needles in an area about 4×4 mm.

In certain embodiments the needles comprising the array can be substantially uniformly distributed. However, in certain embodiments the needles comprising the needle electrode are unevenly distributed. Thus, for example, In certain embodiments the spacing of needles comprising said electrode is denser at the periphery of said electrode and less dense at the center of said electrode, or the spacing of needles comprising the electrode is denser in the center of the electrode and less dense at the periphery of said electrode, or the spacing of needles comprising the electrode increases in density from one edge of the electrode to the opposite edge of the electrode.

In various embodiments the needles are fabricated from a biocompatible metal or combination of materials or its alloy or its oxide. Such metals include, but are not limited to gold, silver, platinum, titanium, chromium, iridium, tungsten, carbon-nanotubes, stainless steel, silver chloride, indium tin oxide (ITO), conductive polymers (Polypyrrole (Ppy) or poly-3,4-ethylenedioxythiophene (PEDOT)) and/or their oxides and/or alloys thereof.

In certain embodiments the needle electrode is configured so that different needles comprising the electrode can be independently stimulated, while in other embodiments the needles are electrically coupled to each other and can be stimulated as a group.

In various embodiments, the needle array is configures so that the electrode array when attached to the skin surface over the spinal cord can stimulate the spinal cord without the use of a conductive gel or cream disposed between the electrode and the skin. In certain embodiments the electrode is configured so that the electrode, when applied to the skin over a region of the spinal cord can conduct a signal having frequency and amplitude sufficient to stimulate the spinal cord without degradation of the electrode.

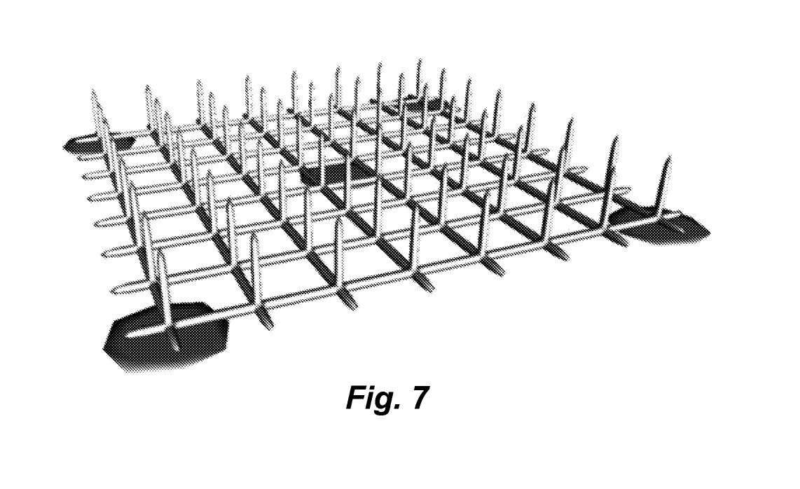

In certain embodiments the needle electrode has hollow grids between the needles comprising the electrode (see, e.g., FIG. 7). In certain embodiments the needle electrode is attached to a conventional transcutaneous electrical stimulation electrode.

In certain embodiments the needle electrode is disposed on a flexible backing, e.g., a polymer backing. Illustrative, but non-limiting polymers include polyimide, parylene, PVC, polyethylene, PEEK, polycarbonate, Ultem PEI, polysulfone, polypropylene, polyurethane, and the like. The backing can optionally comprise a plurality of holes that provide heat and moisture dissipation and/r optionally comprise an adhesive for attachment to the skin surface.

In certain embodiments an electrode array is provided where the electrode array comprises a plurality of needle electrodes, e.g., as described above. In certain embodiments the electrode array comprises at least three needle electrodes, or at least four needle electrodes, or at least 5 needle electrodes, or at least 6 needle electrodes, or at least 7 needle electrodes, or at least 8 needle electrodes, or at least 9 needle electrodes, or at least 10 needle electrodes, or at least 15 needle electrodes, or at least 20 needle electrodes, or at least 25 needle electrodes, or at least 30 needle electrodes, or at least 35 needle electrodes, or at least 40 needle electrodes, or at least 45 needle electrodes, or at least 50 needle electrodes, or at least 75 needle electrodes, or at least 100 needle electrodes. In certain embodiments needle electrodes comprising said array are disposed on a common backing. In certain embodiments the common backing is a rigid backing, while in other embodiments, the common backing is a flexible backing, e.g., a polymer backing. Illustrative, but non-limiting polymers include polyimide, parylene, PVC, polyethylene, PEEK, polycarbonate, Ultem PEI, polysulfone, polypropylene, polyurethane, and the like. The backing can optionally comprise a plurality of holes that provide heat and moisture dissipation and/r optionally comprise an adhesive for attachment to the skin surface.

In certain embodiments different needle electrodes comprising the plurality of needle electrodes in the array are disposed on different backings.

In certain embodiments the different needle electrodes comprising the plurality of needle electrodes in the array are coupled to different electrical leads such that different electrical signals can be applied to different needle electrodes. In certain embodiments one or more electrodes comprising said array are configured to deliver a transcutaneous stimulation signal and one or more electrodes comprising said array are configured to provide a ground or return.

In certain embodiments by way of non-limiting example the needle electrodes comprising the electrode array can be used to record an electrical potential such as an evoked potential from muscle or spinal cord itself or a somatosensory evoked potential.

In various embodiments by way of non-limiting example the needle electrode array may incorporate one or more sensors. One illustrative, but non-limiting sensor is a temperature sensor to monitor the rise in skin temperature associated with the stimulation. Other sensors that may be incorporated into the electrode array may be a flex sensor or pressure sensor to monitor change in position and pressure forces to the skin and electrode itself. In certain embodiments the sensor maybe a photonic sensor use to monitor blood flow.

Temperature sensors (e.g. microthermocouples, thermistors, etc.), flex sensors (e.g., strain gages, rotary encoders, etc.), pressure sensors (e.g., strain gages, piezoelectric crystals, etc.), motion sensors (e.g., accelerometers/gyroscopes), and photonic blood flow sensors are well known to those of skill in the art and are commercially available.

In certain embodiments the needle electrode may be wireless or contain wireless capabilities to communicate freely to the control module and/or other electrodes or sensors.

Also provided are systems for transcutaneous simulation of the spinal cord and/or brain. In various embodiments the systems comprise a needle electrode, e.g., as described above, and/or an electrode array, e.g., as described above, and an electrical stimulator configured to deliver transcutaneous stimulation of the brain or spinal cord through one or more electrodes comprising the electrode array or electrode array assembly. In certain embodiments the system is configured to provide transcutaneous stimulation according to the stimulation parameters described below.

### Fabrication of Needle Electrodes.

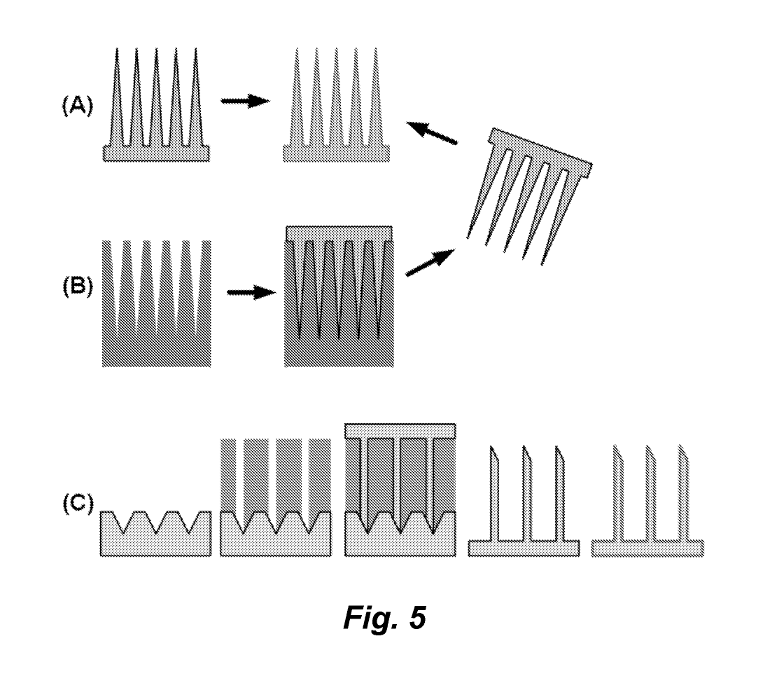

FIG. 5 illustrates various methods to fabricate the needle electrodes described herein. In one approach illustrated in FIG. 5 (A) a needle array is built by 3D printing or laser cutting, and followed with metal encapsulation to provide conductive needle array. In another approach illustrated in FIG. 5 (B) a mold is built by 3D printing or laser cutting, and then a conventional hot-embossing process is utilized to fabricate the array. Finally, metal encapsulation is applied onto the array. In still another approach illustrated in FIG. 5 (C) a substrate with cone-shape holes is prepared. Then a material is deposited onto the substrate with etched tunnel structures leading to the “slanted” surface of the cone-shaped holes. Afterward, another material which is used as electrode substrate is deposited into the tunnel structure. Deposition/encapsulation of biocompatible metal can then applied onto the released electrode for low impedance contact.

### Attachment of Needle Electrodes to the Skin.

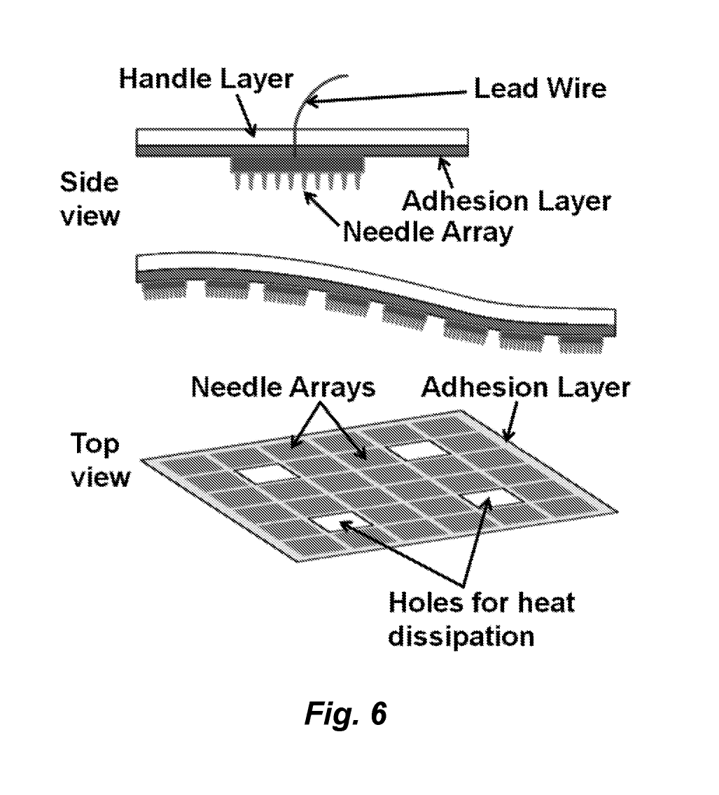

FIG. 6 provides illustrative but non-limiting approach to attachment of the needle electrode(s) to the skin. In practical, the fabricated array can be attached to a conventional transcutaneous electrical stimulation electrode that has an adhesion layer on its surface. Usually, that adhesion layer is also electrical conductive, therefore the lead wire may be able to be eliminated in that case. Note that holes can be created in the transcutaneous electrical stimulation electrode design for heat and moisture dissipation propose. FIG. 7 shows one example of a design of the needle electrode that has hollow grids between needles. After the needle array is attached, e.g., to a conventional transcutaneous electrical stimulation electrode, the sticky layer can be exposed and help the needle array to be attached onto skin.

### Uses of the Electrode Arrays.

Without being bound by a particular theory, it is believed that transcutaneous stimulation, e.g., over one spinal level, over two spinal levels simultaneously, or over three spinal levels simultaneously, optionally in combination with physical training can facilitate recovery of stepping and standing in human subjects following a partial or complete spinal cord injury, a brain injury, or a neurodegenerative pathology. Thus the transcutaneous needle electrodes and/or electrode arrays described herein find use in subjects with a motor incomplete or motor complete spinal cord injury, in subjects with an ischemic brain injury (e.g., from stroke or acute trauma), and in subjects with a neurodegenerative pathology (e.g., stroke, Parkinson's disease, Huntington's disease, Alzheimer's disease, amyotrophic lateral sclerosis (ALS), primary lateral sclerosis (PLS), dystonia, cerebral palsy, and the like).

In addition to the above contexts, the transcutaneous needle electrodes and/or electrode arrays described herein can be utilized in essentially any context where it is desired to deliver a transcutaneous electrical stimulus, e.g., to a tissue.

In some embodiments, the location of electrode(s) in addition to the stimulation parameters may be important in defining the motor response. The use of surface electrode(s), as described herein, facilitates selection or alteration of particular stimulation sites as well as the application of a wide variety of stimulation parameters.

In certain embodiments, the transcutaneous needle electrodes and/or electrode arrays described herein are disposed on the surface of a subject in one or more locations to stimulate the spinal cord (or regions thereof) and thereby activate various central pattern generators and restore endogenous activation patterns to stimulate or improve postural and/or locomotor activity and/or postural or locomotor strength, and/or reaching or grasping and/or hand or upper limb strength, and/or to enable one or more functions such as voluntary voiding of the bladder and/or bowel, sexual function, autonomic control of cardiovascular function, control/regulation of body temperature control, control of digestive functions, control of kidney functions, chewing, swallowing, drinking, talking, or breathing in a normal subject or a subject having a neurologically derived paralysis. The methods typically involve neuromodulating the spinal cord of the subject or a region thereof by administering transcutaneous stimulation to one or more locations on the spinal cord or a region thereof using an electrical stimulator electrically coupled to one or more transcutaneous needle electrodes and/or electrode arrays described herein. In certain embodiments the transcutaneous needle electrodes and/or electrode arrays described herein is disposed over the spinal cord or over one or more regions thereof.

Accordingly, in various embodiments methods and devices are provided to facilitate movement in a mammalian subject (e.g., a human) having spinal cord injury, brain injury, or neurological disease. In certain embodiments the methods involve stimulating the spinal cord of the subject using a transcutaneous needle electrode and/or electrode arrays described herein where the stimulation modulates the electrophysiological properties of selected spinal circuits in the subject so they can be activated, e.g., by proprioceptive derived information and/or input from supraspinal nerves. In various embodiments the stimulation can be accompanied by physical training (e.g., movement) of the region comprising sensory motor circuits involved in the desired motor activity.

In particular illustrative embodiments, the devices and methods described herein stimulate the spinal cord with one or more transcutaneous needle electrodes and/or electrode arrays described herein, that modulate the proprioceptive and/or supraspinal information that controls the lower limbs during standing and/or stepping and/or the upper limbs during reaching and/or grasping conditions. This “sensory” information can guide the activation of the muscles via spinal networks in a coordinated manner and in a manner that accommodates the external conditions, e.g., the amount of loading, speed and direction of stepping or whether the load is equally dispersed on the two lower limbs, indicating a standing event, alternating loading indicating stepping, or sensing postural adjustments signifying the intent to reach and grasp.

Unlike approaches that involve specific stimulation of motor neurons to directly induce a movement, the methods described herein enable the spinal circuitry to control the movements. More specifically, the devices and methods described herein exploit the spinal circuitry and its ability to interpret proprioceptive and/or cutaneous information and to respond to that proprioceptive and/or cutaneous information in a functional way. For example, the human spinal cord can receive sensory input associated with a movement such as stepping, and this sensory information can be used to modulate the motor output to accommodate the appropriate speed of stepping and level of load that is imposed on lower limbs. In some embodiments, the present methods can utilize the central-pattern-generation-like properties of the human spinal cord (e.g., the lumbosacral spinal cord, the thoracic spinal cord, the cervical spinal cord). Thus, for example, exploiting inter alia the central-pattern-generation-like properties of the lumbosacral spinal cord, oscillations of the lower limbs can be induced simply by vibrating the vastus lateralis muscle of the lower limb, and/or by transcutaneous stimulation of the spinal cord and/or ganglia, and/or by stretching the hip. The methods described herein exploit the fact that the human spinal cord, in complete or incomplete SCI subjects, can receive and interpret proprioceptive and somatosensory information that can be used to control the patterns of neuromuscular activity among the motor pools necessary to generate particular movements, e.g., standing, stepping, reaching, grasping, and the like. In various embodiments this is in contrast to other approaches where the actual movement is induced/controlled by direct stimulation (e.g., of particular motor neurons and/or muscles).

In one illustrative embodiment, the subject is fitted with one or more transcutaneous needle electrodes and/or electrode arrays described herein that afford selective stimulation and control capability to select sites, mode(s), and intensity of stimulation via electrodes placed over, for example, the lumbosacral spinal cord, and/or the thoracic spinal cord, and/or cervical spinal cord to facilitate movement of the arms and/or legs of individuals with a spinal cord injury or another severely debilitating neuromotor disorder.

In certain embodiments the transcutaneous needle electrode and/or electrode array described herein can be disposed on the surface of the subject and typically the subject can be immediately tested to identify the most effective subject specific stimulation paradigms for facilitation of movement (e.g., stepping and standing and/or arm and/or hand movement). In certain embodiments, using these stimulation paradigms the subject can practice standing and stepping and/or reaching or grabbing in an interactive rehabilitation program while being subject to spinal stimulation.

Depending on the site/type of injury and the locomotor and motor activity it is desired to facilitate particular spinal stimulation protocols include, but are not limited to specific stimulation sites along the lumbosacral and/or thoracic, and/or cervical spinal cord; specific combinations of stimulation sites along the lumbosacral and/or thoracic, and/or cervical spinal cord; specific stimulation amplitudes; specific stimulation polarities (e.g., monopolar and bipolar stimulation modalities); specific stimulation frequencies; and/or specific stimulation pulse widths.

In various embodiments, the methods described herein can comprise transcutaneous stimulation of one or more regions of the spinal cord and/or brain, and/or brain stem in combination with locomotor or motor activities thereby providing modulation of the electrophysiological properties of spinal circuits in the subject so they are activated by proprioceptive information derived from the region of the subject where locomotor or motor activity is to be facilitated. Further, spinal stimulation in combination with pharmacological agents and locomotor or motor activity may result in the modulation of the electrophysiological properties of spinal circuits in the subject so they are activated by proprioceptive information derived from the region of the subject where locomotor or motor activity is to be facilitated.

In certain embodiments locomotor activity of the region of interest can be assisted or accompanied by any of a number of methods known, for example, to physical therapists. By way of illustration, individuals after severe SCI can generate standing and stepping patterns when provided with body weight support on a treadmill and manual assistance. During both stand and step training of human subjects with SCI, the subjects can be placed on a treadmill in an upright position and suspended in a harness at the maximum load at which knee buckling and trunk collapse can be avoided. Trainers positioned, for example, behind the subject and at each leg assist as needed in maintaining proper limb kinematics and kinetics appropriate for each specific task. During bilateral standing, both legs can be loaded simultaneously and extension can be the predominant muscular activation pattern, although co-activation of flexors can also occur. Additionally, or alternatively, during stepping the legs can be loaded in an alternating pattern and extensor and flexor activation patterns within each limb also alternated as the legs moved from stance through swing. Afferent input related to loading and stepping rate can influence these patterns, and training has been shown to improve these patterns and function in clinically complete SCI subjects.

Transcutaneous Stimulation of a Region of the Cervical Spine

In various embodiments, the methods described herein involve transcutaneous electrical stimulation of the cervical spinal cord or a region of the cervical spinal cord of the subject utilizing one or more of the transcutaneous needle electrodes and/or electrode arrays described herein. Illustrative regions include, but are not limited to, one or more regions straddling or spanning a region selected from the group consisting of C0-C1, C0-C2, C0-C3, C0-C4, C0-C5, C0-C6, C0-C7, C1-C1, C1-C2, C1-C3, C1-C4, C1-C7, C1-C6, C1-C7, C1-T1, C2-C2, C2-C3, C2-C4, C2-C5, C2-C6, C2-C7, C2-T1, C3-C3, C3-C4, C3-05, C3-C6, C3-C7, C3-T1, C4-C4, C4-C5, C4-C6, C4-C7, C4-T1, C5-C5, C5-C6, C5-C7, C5-T1, C6-C6, C6-C7, C6-T1, C7-C7, and C7-T1.

Transcutaneous Stimulation of a Region of the Thoracic Spine

In various embodiments, the methods described herein involve transcutaneous electrical stimulation of the thoracic spinal cord or a region of the thoracic spinal cord of the subject utilizing one or more of the transcutaneous needle electrodes and/or electrode arrays described herein. Illustrative regions include, but are not limited to, one or more regions straddling or spanning a region selected from the group consisting of T1-T1, T1-T2, T1-T3, T1-T4, T1-T5, T1-T6, T1-T7, T1-T8, T1-T9, T1-T10, T1-T11, T1-T12, T2-T1, T2-T2, T2-T3, T2-T4, T2-T5, T2-T6, T2-T7, T2-T8, T2-T9, T2-T10, T2-T11, T2-T12, T3-T1, T3-T2, T3-T3, T3-T4, T3-T5, T3-T6, T3-T7, T3-T8, T3-T9, T3-T10, T3-T11, T3-T12, T4-T1, T4-T2, T4-T3, T4-T4, T4-T5, T4-T6, T4-T7, T4-T8, T4-T9, T4-T10, T4-T11, T4-T12, T5-T1, T5-T2, T5-T3, T5-T4, T5-T5, T5-T6, T5-T7, T5-T8, T5-T9, T5-T10, T5-T11, T5-T12, T6-T1, T6-T2, T6-T3, T6-T4, T6-T5, T6-T6, T6-T7, T6-T8, T6-T9, T6-T10, T6-T11, T6-T12, T7-T1, T7-T2, T7-T3, T7-T4, T7-T5, T7-T6, T7-T7, T7-T8, T7-T9, T7-T10, T7-T11, T7-T12, T8-T1, T8-T2, T8-T3, T8-T4, T8-T5, T8-T6, T8-T7, T8-T8, T8-T9, T8-T10, T8-T11, T8-T12, T9-T1, T9-T2, T9-T3, T9-T4, T9-T5, T9-T6, T9-T7, T9-T8, T9-T9, T9-T10, T9-T11, T9-T12, T10-T1, T10-T2, T10-T3, T10-T4, T10-T5, T10-T6, T10-T7, T10-T8, T10-T9, T10-T10, T10-T11, T10-T12, T11-T1, T11-T2, T11-T3, T11-T4, T11-T5, T11-T6, T11-T7, T11-T8, T11-T9, T11-T10, T11-T11, T11-T12, T12-T1, T12-T2, T12-T3, T12-T4, T12-T5, T12-T6, T12-T7, T12-T8, T12-T9, T12-T10, T12-T11, T12-T12, T12-L1, and L5 to S1.

Transcutaneous Stimulation of the Lumbosacral Spinal Cord.

In various embodiments, the methods described herein involve transcutaneous electrical stimulation of the lumbosacral spinal cord or a region of the lumbosacral spinal cord of the subject utilizing one or more of the transcutaneous needle electrodes and/or electrode arrays described herein. Illustrative regions include, but are not limited to, one or more regions straddling or spanning a region selected from the group consisting of L1-L1, L1-L2, L1-L3, L1-L4, L1-L5, L2-L1, L2-L2, L2-L3, L2-L4, L2-L5, L3-L1, L3-L2, L3-L3, L3-L4, L3-L5, L4-L1, L4-L2, L4-L3, L4-L4, L4-L5, L5-L1, L5-L2, L5-L3, L5-L4, L5-L5, L5-S1.

Transcutaneous Stimulation Parameters.

In certain embodiments, the transcutaneous al stimulation is at a frequency ranging from about 0.5 Hz, or 3 Hz, or from about 5 Hz, or from about 10 Hz up to about 50 kHz, or up to about 30 kHz, or up to about 20 kHz, or up to about 10 kHz, or up to about 1,000 Hz, or up to about 500 Hz, or up to about 100 Hz, or up to about 80 Hz, or up to about 40 Hz, or from about 3 Hz or from about 5 Hz up to about 80 Hz, or from about 5 Hz up to about 30 Hz, or up to about 40 Hz, or up to about 50 Hz.

In certain embodiments, the transcutaneous stimulation is applied at an intensity (amplitude) ranging from about 10 mA up to about 500 mA, or up to about 300 mA, or up to about 150 mA, or from about 20 mA up to about 300 mA, or up to about 50 mA or up to about 100 mA, or from about 20 mA or from about 30 mA, or from about 40 mA up to about 50 mA, or up to about 60 mA, or up to about 70 mA or up to about 80 mA.

In certain embodiments, the pulse width ranges from about 100 μs up to about 1000 μs, or from about 150 μs up to about 600 μs, or from about 200 μs up to about 500 μs, or from about 200 μs to about 450 μs.

In certain embodiments the stimulation pulse is delivered superimposed on a high frequency carrier signal. In certain embodiments the high frequency ranges from about 3 kHz, or about 5 kHz, or about 8 kHz up to about 100 kHz, or up to about 80 kHz, or up to about 50 kHz, or up to about 40 kHz, or up to about 30 kHz, or up to about 20 kHz, or up to about 15 kHz. In certain embodiments the carrier frequency amplitude ranges from about 30 mA, or about 40 mA, or about 50 mA, or about 60 mA, or about 70 mA, or about 80 mA up to about 500 mA, or up to about 400 mA, or up to about 300 mA, or up to about 200 mA, or up to about 150 mA.

In one illustrative, but non-limiting embodiment, a bipolar rectangular stimuli (1-msec duration) with a carrier frequency of 10 kHz and at intensities ranging from 30 to 300 mA is used. The stimulation can be at 5 Hz, for example, with an illustrative, but non-limiting exposure duration ranging from 10 to 30 sec. An illustrative, but non-limiting signal intensity is from about 80 mA, or from about 100 mA, or from about 110 mA to about 200 mA, or to about 180 mA, or to about 150 mA.

In certain embodiments the transcutaneous stimulation is at a frequency and amplitude sufficient to stimulate or improve postural and/or locomotor activity and/or postural or locomotor strength. In certain embodiments the transcutaneous stimulation is at a frequency and amplitude sufficient to stimulate or improve postural and/or locomotor activity and/or postural or locomotor strength when applied in conjunction with a neuromodulatory agent (e.g., a monoaminergic agent). In certain embodiments the transcutaneous stimulation is at a frequency and amplitude sufficient to stimulate grasping, and/or improve hand strength and/or fine hand control. In certain embodiments the transcutaneous stimulation is at a frequency and amplitude sufficient to improve stimulate grasping and/or to improve hand strength and/or fine hand control when applied in conjunction with a neuromodulatory agent (e.g., a monoaminergic agent). In certain embodiments the transcutaneous stimulation is at a frequency and amplitude sufficient to stimulate voluntary voiding of the bladder and/or bowel, and/or return of sexual function, and/or autonomic control of cardiovascular function, and/or body temperature, control of digestive functions, control of kidney functions, chewing, swallowing, drinking, talking, or breathing in a normal subject or a subject having a neurologically derived paralysis. In certain embodiments the transcutaneous stimulation is at a frequency and amplitude sufficient to stimulate voluntary voiding of the bladder and/or bowel, and/or return of sexual function, and/or autonomic control of cardiovascular function, and/or body temperature when applied in conjunction with a neuromodulatory agent (e.g., a monoaminergic agent). In certain embodiments the carrier frequency, when present, is at frequency and intensity sufficient to minimize subject discomfort.

By way of illustration, non-invasive transcutaneous electrical spinal cord stimulation (tSCS) can induce locomotor or motor-like activity in non-injured humans. Continuous tSCS (e.g., at 5-40 Hz) applied paraspinally over the T11-T12 vertebrae can induce involuntary stepping movements in subjects with their legs in a gravity-independent position. These stepping movements can be enhanced when the spinal cord is stimulated at two to three spinal levels (C5, T12, and/or L2) simultaneously with frequency in the range of 5-40 Hz. Further, locomotion of can be improved, in some embodiments substantially, when locomotor and postural spinal neuronal circuitries are stimulated simultaneously.

In another illustrative, but non-limiting embodiment transcutaneous electrical stimulation (5 Hz) delivered simultaneously at the C5, T11, and L2 vertebral levels facilitated involuntary stepping movements that were significantly stronger than stimulation at T11 alone. Accordingly, simultaneous spinal cord stimulation at multiple sites can have an interactive effect on the spinal circuitry responsible for generating locomotion.

International Patent Publication No: WO/2012/094346 demonstrates that locomotor activity and/or strength and/or posture can be improved and/or restored by stimulation of the spinal circuitry. The methods described in WO/2012/094346 can be further enhanced by the use of the improved transcutaneous electrode arrays described herein.

With respect to hand control, it is noted that WO/2015/048563 (PCT/US2014/057886) shows that the cervical spinal cord can be neuromodulated using two paradigms, i.e., electrically and pharmacologically. Moreover, the data presented therein indicate that non-functional networks can become engaged and progressively improve motor performance. In addition, the further improvement in hand function after withdrawing painless cutaneous Enabling motor control (pcEmc) and pharmacological Enabling motor control *(fEmc) suggests that once functional connections are established they remain active. The methods described in WO/2015/048563 can be further enhanced by the use of the improved transcutaneous electrode arrays described herein.

Application of Transcutaneous Electrode Arrays.

As noted above, the transcutaneous electrode array may applied to the surface of a body using any of a number of methods well known to those of skill in the art.

In one embodiment, the subject is fitted with one or more transcutaneous needle electrodes and/or electrode arrays described herein that afford selective stimulation and control capability to select sites, mode(s), and intensity of stimulation via electrodes placed superficially over, for example, the lumbosacral spinal cord and/or the thoracic spinal cord, and/or the cervical spinal cord to facilitate movement of the arms and/or legs of individuals with a severely debilitating neuromotor disorder.

In some embodiments, the subject is provided a generator control unit and is fitted with an electrode(s) and then tested to identify the most effective subject specific stimulation paradigms for facilitation of movement (e.g., stepping and standing and/or arm and/or hand movement). Using the herein described stimulation paradigms, the subject practices standing, stepping, reaching, grabbing, breathing, and/or speech therapy in an interactive rehabilitation program while being subject to spinal stimulation.

Depending on the site/type of injury and the locomotor or motor activity it is desired to facilitate, particular spinal stimulation protocols include, but are not limited to, specific stimulation sites along the lumbosacral, thoracic, cervical spinal cord or a combination thereof; specific combinations of stimulation sites along the lumbosacral, thoracic, cervical spinal cord and/or a combination thereof; specific stimulation amplitudes; specific stimulation polarities (e.g., monopolar and bipolar stimulation modalities); specific stimulation frequencies; and/or specific stimulation pulse widths.

In various embodiments, the system is designed so that the patient can use and control it in the home environment.

In various embodiments, transcutaneous needle electrodes and/or electrode arrays described herein are operably linked to control circuitry that permits selection of electrode(s) to activate/stimulate and/or that controls frequency, and/or pulse width, and/or amplitude of stimulation. In various embodiments, the electrode selection, frequency, amplitude, and pulse width are independently selectable, e.g., at different times, different electrodes can be selected. At any time, different electrodes can provide different stimulation frequencies and/or amplitudes. In various embodiments, different electrodes or all electrodes can be operated in a monopolar mode and/or a bipolar mode, using, e.g., constant current or constant voltage delivery of the stimulation.

It will be recognized that any present or future developed stimulation system capable of providing an electrical signal to one or more regions of the spinal cord may be used in accordance with the teachings provided herein.

In one illustrative but non-limiting system a control module is operably coupled to a signal generation module and instructs the signal generation module regarding the signal to be generated. For example, at any given time or period of time, the control module may instruct the signal generation module to generate an electrical signal having a specified pulse width, frequency, intensity (current or voltage), etc. The control module may be preprogrammed prior to use or receive instructions from a programmer (or another source). Thus, in certain embodiments the pulse generator/controller is configurable by software and the control parameters may be programmed/entered locally, or downloaded as appropriate/necessary from a remote site.

In certain embodiments the pulse generator/controller may include or be operably coupled to memory to store instructions for controlling the stimulation signal(s) and may contain a processor for controlling which instructions to send for signal generation and the timing of the instructions to be sent.

While in certain embodiments, two leads are utilized to provide transcutaneous stimulation, it will be understood that any number of one or more leads may be employed. In addition, it will be understood that any number of one or more electrodes per lead may be employed. Stimulation pulses are applied to transcutaneous needle electrodes and/or electrode arrays described herein (which typically are cathodes) with respect to a return electrode (which typically is an anode) to induce a desired area of excitation of electrically excitable tissue in one or more regions of the spine. A return electrode such as a ground or other reference electrode can be located on the same lead as a stimulation electrode. However, it will be understood that a return electrode may be located at nearly any location, whether in proximity to the stimulation electrode or at a more remote part of the body, or as part of a metallic case such as at a metallic case of a pulse generator. It will be further understood that any number of one or more return electrodes may be employed. For example, there can be a respective return electrode for each cathode such that a distinct cathode/anode pair is formed for each cathode.

In various embodiments, the approach is not to electrically induce a walking pattern, standing pattern, or moving pattern of activation, but to enable/facilitate it so that when the subject manipulates their body position, the spinal cord can receive proprioceptive information from the legs (or arms) that can be readily recognized by the spinal circuitry. Then, the spinal cord knows whether to step or to stand or to reach or to grasp, or to do nothing. In other words, this enables the subject to begin stepping or to stand or to reach and grasp when they choose after the stimulation pattern has been initiated.

Moreover, the methods and devices described herein are effective in a spinal cord injured subject that is clinically classified as motor complete; that is, there is no motor function below the lesion. In various embodiments, the specific combination of electrode(s) activated/stimulated and/or the desired stimulation of any one or more electrodes and/or the stimulation amplitude (strength) can be varied in real time, e.g., by the subject. Closed loop control can be embedded in the process by engaging the spinal circuitry as a source of feedback and feedforward processing of proprioceptive input and by voluntarily imposing fine tuning modulation in stimulation parameters based on visual, and/or kinetic, and/or kinematic input from selected body segments.

In various embodiments, the devices, optional pharmacological agents, and methods are designed so that a subject with no voluntary movement capacity can execute effective standing and/or stepping and/or reaching and/or grasping. In addition, the approach described herein can play an important role in facilitating recovery of individuals with severe although not complete injuries.

The approach described herein can provide some basic postural, locomotor and reaching and grasping patterns on their own. However, in some embodiments, the methods described herein can also serve as building blocks for future recovery strategies. In other embodiments, combining transcutaneous stimulation of appropriate spinal circuits with physical rehabilitation and pharmacological intervention can provide practical therapies for complete SCI human patients. The methods described herein can be sufficient to enable weight bearing standing, stepping and/or reaching or grasping in SCI patients. Such capability can give SCI patients with complete paralysis or other neuromotor dysfunctions the ability to participate in exercise, which can be beneficial, if not highly beneficial, for their physical and mental health.

In other embodiments, the methods described herein can enable movement with the aid of assistive walkers and/or robotic devices or systems including, but not limited to an exoskeletal system and any robotic prosthetic device on extremity or trunk. In some embodiments, simple standing and short duration walking can increase these patients' autonomy and quality of life. The stimulating technology described herein (e.g., transcutaneous electrical stimulation) can provide a direct brain-to-spinal cord interface that can enable more lengthy and finer control of movements.

The transcutaneous electrode stimulation systems described herein are intended to be illustrative and non-limiting. Using the transcutaneous needle electrodes and/or electrode arrays described herein, fabrication methods, and teachings provided herein, alternative transcutaneous stimulation systems and methods will be available to one of skill in the art.

### Use of Neuromodulatory Agents.

In certain embodiments, the transcutaneous stimulation methods described herein are used in conjunction with various pharmacological agents, particularly pharmacological agents that have neuromodulatory activity (e.g., that are monoamergic). In certain embodiments, the use of various serotonergic, and/or dopaminergic, and/or noradrenergic, and/or GABAergic, and/or glycinergic drugs is contemplated. These agents can be used in conjunction with transcutaneous stimulation and/or physical therapy as described above. This combined approach can help to put the spinal cord in an optimal physiological state for controlling a range of hand and/or upper limb movements or lower limb movements or for regulating posture, and the like.

In certain embodiments, the drugs are administered systemically, while in other embodiments, the drugs are administered locally, e.g., to particular regions of the spinal cord. Drugs that modulate the excitability of the spinal neuromotor networks include, but are not limited to combinations of noradrenergic, serotonergic, GABAergic, and glycinergic receptor agonists and antagonists.

Dosages of at least one drug or agent can be between about 0.001 mg/kg and about 10 mg/kg, between about 0.01 mg/kg and about 10 mg/kg, between about 0.01 mg/kg and about 1 mg/kg, between about 0.1 mg/kg and about 10 mg/kg, between about 5 mg/kg and about 10 mg/kg, between about 0.01 mg/kg and about 5 mg/kg, between about 0.001 mg/kg and about 5 mg/kg, or between about 0.05 mg/kg and about 10 mg/kg. Typically where the drug is an approved drug, it will be administered at dosage consistent with the recommended/approved dosage for that drug.

Drugs or agents can be delivery by injection (e.g., subcutaneously, intravenously, intramuscularly), orally, rectally, or inhaled.

Illustrative pharmacological agents include, but are not limited to, agonists and antagonists to one or more combinations of serotonergic: 5-HT1A, 5-HT2A, 5-HT3, and 5HT7 receptors; to noradrenergic alpha 1 and 2 receptors; and to dopaminergic D1 and D2 receptors (see, e.g., Table 1).

TABLE 1
Illustrative, but non-limiting pharmacological agents.

| | | | | | |
|---|---|---|---|---|---|
| | | | | Typical | Typical |
| | | | | Dose | Range |
| Name | Target | Action | Route | (mg/Kg) | (mg/kg) |

Serotonergic receptor systems

| | | | | | |
|---|---|---|---|---|---|
| 8-OHDPAT | 5-HT1A7 | Agonist | S.C. | 0.05 | 0.045-0.3 |
| Way 100.635 | 5-HT1A | Antagonist | I.P. | 0.5 | 0.4-1.5 |
| Quipazine | 5-HT2A/C | Agonist | I.P. | 0.2 | 0.18-0.6 |
| Ketanserin | 5-HT2A/C | Antagonist | I.P. | 3 | 1.5-6.0 |
| SR 57227A | 5-HT3 | Agonist | I.P. | 1.5 | 1.3-1.7 |
| Ondanesetron | 5-HT3 | Antagonist | I.P. | 3 | 1.4-7.0 |
| SB269970 | 5-HT7 | Antagonist | I.P. | 7 | 2.0-10.0 |
| Buspirone | | | | | |

Noradrenergic receptor systems

| | | | | | |
|---|---|---|---|---|---|
| Methoxamine | Alpha1 | Agonist | I.P. | 2.5 | 1.5-4.5 |
| Prazosin | Alpha1 | Antagonist | I.P. | 3 | 1.8-3.0 |
| Clonidine | Alpha2 | Agonist | I.P. | 0.5 | 0.2-1.5 |
| Yohimbine | Alpha2 | Antagonist | I.P. | 0.4 | 0.3-0.6 |

Dopaminergic receptor systems

| | | | | | |
|---|---|---|---|---|---|
| SKF-81297 | D1-like | Agonist | I.P. | 0.2 | 0.15-0.6 |
| SCH-23390 | D1-like | Antagonist | I.P. | 0.15 | 0.1-0.75 |
| Quinipirole | D2-like | Agonist | I.P. | 0.3 | 0.15-0.3 |
| Eticlopride | D2-like | Antagonist | I.P. | 1.8 | 0.9-1.8 |

In certain embodiments the neuromodulatory agent (drug) is a molecule that activates (e.g., selectively activates) an α2c adrenergic receptor subtype and/or that blocks (e.g., selectively blocks) blocking an α2a adrenergic receptor subtype. In certain embodiments the molecule that activates an α2c adrenergic receptor subtype is 2-[(4,5-Dihydro-1H-imidazol-2-yl)methyl]-2,3-dihydro-1-methyl-1H-isoindole (BRL-44408). In certain embodiments the molecule that activates an α2c adrenergic receptor subtype is (R)-3-nitrobiphenyline and/or a compound according to the formula:

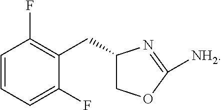 [SMILES: NC1=N[C@@H](Cc2c(F)cccc2F)CO1]

In certain embodiments the neuromodulatory agent comprises Clonidine. In certain embodiments the neuromodulatory agent further comprises a 5-HT1 and/or a 5-HT7 serotonergic agonist.

In certain embodiments the neuromodulatory includes any neuromodulatory agent or combination of agents described in US 2016/0158204 A1 which is incorporated herein by reference for the neuromodulatory agents and combinations thereof described therein.

The foregoing methods are intended to be illustrative and non-limiting. Using the teachings provided herein variations on these embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

### REFERENCES

[1] Ward and Robertson (1998) Sensory, motor, and pain thresholds for stimulation with medium frequency alternating current, Arch. Phys. Med. Rehab., 79: 273-278.
[2] Wiley and Webster (1982) Analysis and Control of the Current Distribution under Circular Dispersive Electrodes, Biomed. Engin., IEEE Trans. BME-29: 381-385.
[3] Bruckenstein and Miller (1970) An Experimental Study of Nonuniform Current Distribution at Rotating Disk Electrodes, J. Electrochem. Soc., 117: 1044-1048.
[4] Rubinstein et al. (1987) Current Density Profiles of Surface Mounted and Recessed Electrodes for Neural Prostheses, Biomed. Engin., IEEE Trans. BME-34: 864-875.
[5] Yongmin and Schimpf (1996) Electrical behavior of defibrillation and pacing electrodes, Proc. Ieee, 84: 446-456.
[6] Tungjitkusolmun et al. (2000) Finite element analysis of uniform current density electrodes for radio-frequency cardiac ablation, Ieee Trans. Biomed. Engin., 47: 32-40.
[7] Valchinov and Pallikarakis (2004) An active electrode for biopotential recording from small localized bio-sources, BioMed. Engin. OnLine, 3: 25, 2004.
[8] Chen-Chun et al. (2010) Transparent microprobe array fabricated by MEMS hot embossing technology for photodynamic therapy application, IEICE Electron. Exp., 7: 569-576.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A needle electrode for transcutaneous neural stimulation, said electrode comprising:
a plurality of electrically conductive needles, wherein said needles are solid, or wherein said needles are hollow and have a closed tip, wherein said needles having an average tip diameter less than about 10 μm and an average length greater than about 10 μm or greater than about 20 μm wherein said electrically conductive needles are electrically coupled to one or more electrical leads.

2. The needle electrode of claim 1, wherein said needles are solid.

3. The needle electrode of claim 1, wherein said needles are hollow and have a closed tip.

4. The needle electrode according to any one of claims 1-3, wherein said electrode comprises at least about 10 needles, or at least about 15 needles, or at least about 20 needles, or at least about 25 needles, or at least about 30 needles, or at least about 40 needles, or at least about 50 needles, or at least about 100 needles, or at least about 200 needles, or at least about 300 needles, or at least about 400 needles, or at least about 500 needles, or at least about 600 needles, or at least about 700 needles, or at least about 800 needles, or at least about 900 needles, or at least about 1000 needles.

5. The needle electrode according to any one of claims 1-4, wherein said needles are of sufficient length to penetrate at least 70%, or at least 80%, or at least 90%, or at least 100% through the stratum corneum of the skin when the electrode is attached to the surface of a human over the spinal cord.

6. The needle electrode according to any one of claims 1-5, wherein the needles are of a length that does not substantially penetrate subcutaneous tissue below the stratum corneum.

7. The needle electrode according to any one of claims 1-5, wherein the average length of said ranges from about 1 μm up to about 100 μm, or from about 1 μm up to about 80 μm, or from about 1 μm up to about 50 μm, or from about 1 μm up to about 30 μm, or from about 1 μm up to about 20 μm, or is at least about 30 μm, or at least about 40 μm, or at least about 50 μm, or at least about 60 μm, or at least about 70 μm.

8. The needle electrode according to any one of claims 1-7, the average length of said needles is less than about 200 μm, or less than about 150 μm, or less than about 100 μm.

9. The needle electrode according to any one of claims 1-5, wherein the average length of said needles ranges from about 40 to about 60 μm.

10. The needle electrode according to any one of claims 1-5, wherein the average length of said needles is about 50 μm.

11. The needle electrode according to any one of claims 1-10, wherein the tip of said needles ranges in diameter (or maximum cross-sectional dimension) from about 0.1 μm up to about 10 μm, or from about 0.5 μm up to about 6 μm, or from about 1 μm up to about 4 μm.

12. The needle electrode according to any one of claims 1-11, wherein the average separation between two adjacent needles ranges from about 0.01 mm up to about 1 mm, or about 0.05 mm up to about 0.5 mm, or about 0.1 mm up to about 0.4 mm, or up to about 0.3 mm, or up to about 0.2 mm.

13. The needle electrode according to any one of claims 1-12, wherein the average separation between two adjacent needles ranges from about 0.15 mm up to about 0.25 mm.

14. The needle electrode according to any one of claims 1-13, wherein said needles are disposed in an area of about 1 cm² or less, or about 0.8 cm² or less, or about 0.6 cm² or less, or about 0.5 cm² or less, or about 0.4 cm² or less, or about 0.3 cm² or less, or about 0.2 cm² or less, or about 0.1 cm² or less.

15. The needle electrode according to any one of claims 1-14, wherein said needles are disposed in an area of about 2 mm or about 3 mm, or about 4 mm, or about 5 mm, or about 6 mm, or about 7 mm or about 8 mm, or about 9 mm, or about 10 mm by about 2 mm or about 3 mm, or about 4 mm, or about 5 mm, or about 6 mm, or about 7 mm or about 8 mm, or about 9 mm, or about 10 mm.

16. The needle electrode according to any one of claims 1-14, wherein said electrode comprises about 20×about 20 needles in an area about 4×4 mm.

17. The needle electrode according to any one of claims 1-16, wherein the needles comprising said electrode are substantially uniformly distributed.

18. The needle electrode according to any one of claims 1-17, wherein the needles comprising said needle electrode are unevenly distributed.

19. The needle electrode of claim 18, wherein the spacing of needles comprising said electrode is denser at the periphery of said electrode and less dense at the center of said electrode.

20. The needle electrode of claim 18, wherein the spacing of needles comprising said electrode is denser in the center of the electrode and less dense at the periphery of said electrode.

21. The needle electrode of claim 18, wherein the spacing of needles comprising said electrode increases in density from one edge of the electrode to the opposite edge of the electrode.

22. The needle electrode according to any one of claims 1-21, wherein said electrode at 10 kHz stimulation frequency has an electrode skin impedance less than ½ the electrode skin impedance of a flat silver chloride (AgCl) electrode having the same projected area.

23. The needle electrode according to any one of claims 1-22, wherein a micro-needle array with 20×20 needles in a 4×4 mm² electrode unit provides an electrode-skin interface impedance at 10 kHz stimulation frequency, of less than about 0.5 Ω/cm2, or less than about 0.249 Ω/cm2.

24. The needle electrode according to any one of claims 1-23, wherein said needles are fabricated from a material selected from the group consisting of platinum, titanium, chromium, iridium, tungsten, gold, carbon nanotubes, stainless steel, silver, silver chloride, indium tin oxide (ITO), conductive polymers (Polypyrrole (Ppy) or poly-3,4-ethylenedioxythiophene (PEDOT)).

25. The needle electrode according to any one of claims 1-23, wherein said needles are fabricated from a material selected from the group consisting of platinum, titanium, chromium, iridium, tungsten, gold, stainless steel, silver, tin, indium, indium tin oxide, oxides thereof, nitrides thereof, and alloys thereof.

26. The needle electrode according to any one of claims 1-25, wherein different needles comprising said electrode can be independently stimulated.

27. The needle electrode according to any one of claims 1-25, wherein said needles are electrically coupled to each other and can be stimulated as a group.

28. The needle electrode according to any one of claims 1-27, wherein said electrode array when attached to the skin surface over the spinal cord can stimulate the spinal cord without the use of a conductive gel or cream disposed between the electrode and the skin.

29. The needle electrode according to any one of claims 1-28, wherein said electrode, when applied to the skin over a region of the spinal cord can conduct a signal having frequency and amplitude sufficient to stimulate the spinal cord without degradation of the electrode.

30. The needle electrode according to any one of claims 1-29, wherein said needle electrode has hollow grids between the needles comprising said electrode.

31. The needle electrode according to any one of claims 1-30, wherein said needle electrode is attached to a conventional transcutaneous electrical stimulation electrode.

32. The needle electrode according to any one of claims 1-31, wherein said electrode is disposed on a flexible backing.

33. The needle electrode of claim 32, wherein said flexible backing comprises a polymer.

34. The needle electrode of claim 33, wherein said flexible backing comprise a polymer selected from the group consisting of polyimide, parylene, PVC, polyethylene, PEEK, polycarbonate, Ultem PEI, polysulfone, polypropylene, and polyurethane.

35. The needle electrode according to any one of claims 32-34, wherein said backing comprises a plurality of holes that provide heat and moisture dissipation.

36. The needle electrode according to any one of claims 32-35, wherein said backing comprises an adhesive for attachment to the skin surface.

37. An electrode array comprising a plurality of needle electrodes according to any one of claims 1-36.

38. The electrode array of claim 37, wherein said electrode array comprises at least three needle electrodes, or at least four needle electrodes, or at least 5 needle electrodes, or at least 6 needle electrodes, or at least 7 needle electrodes, or at least 8 needle electrodes, or at least 9 needle electrodes, or at least 10 needle electrodes, or at least 15 needle electrodes, or at least 20 needle electrodes, or at least 25 needle electrodes, or at least 30 needle electrodes, or at least 35 needle electrodes, or at least 40 needle electrodes, or at least 45 needle electrodes, or at least 50 needle electrodes, or at least 75 needle electrodes, or at least 100 needle electrodes.

39. The electrode array according to any one of claims 37-38, wherein said needle electrodes are disposed on a common backing.

40. The electrode array of claim 39, wherein said common backing is a flexible backing.

41. The electrode array of claim 40, wherein said flexible backing comprises a polymer.

42. The electrode array of claim 41, wherein said flexible backing comprise a polymer selected from the group consisting of polyimide, parylene, PVC, polyethylene, PEEK, polycarbonate, Ultem PEI, polysulfone, polypropylene, and polyurethane.

43. The electrode array according to any one of claims 39-42, wherein said common backing comprises a plurality of holes that provide heat and moisture dissipation.

44. The electrode array according to any one of claims 39-43, wherein said common backing comprises an adhesive for attachment to the skin surface.

45. The electrode array according to any one of claims 37-38, wherein different needle electrodes comprising said plurality of needle electrodes are disposed on different backings.

46. The electrode array according to any one of claims 37-45, wherein different needle electrodes comprising said plurality of needle electrodes are coupled to different electrical leads such that different electrical signals can be applied to different needle electrodes.

47. The electrode array according to any one of claims 37-46, wherein one or more electrodes comprising said array are configured to deliver a transcutaneous stimulation signal and one or more electrodes comprising said array are configured to provide a ground or return.

48. The electrode array according to any one of claims 37-47, wherein one or more needle electrodes is configured to record an electrical potential.

49. The electrode array according to any one of claims 37-48, wherein the electrode array is wireless or contains wireless capabilities.

50. A system for transcutaneous simulation of the spinal cord and/or brain, said system comprising:
a needle electrode according to any one of claims 1-36 or an electrode array according to any one of claims 37-49; and
an electrical stimulator configured to deliver transcutaneous stimulation of the brain or spinal cord through one or more electrodes comprising said electrode array or electrode array assembly.

51. The system of claim 50, wherein said system is configured to provide transcutaneous stimulation signal at a frequency ranging from about 0.3 Hz, or from about 1H, or from about 5 Hz, or from about 10 Hz up to about 50 kHz, or up to about 30 kHz, or up to about 20 kHz, or up to about 10 kHz, or up to about 1,000 Hz, or up to about 500 Hz, or up to about 100 Hz, or up to about 80 Hz, or up to about 40 Hz, or from about 3 Hz or from about 5 Hz up to about 80 Hz, or from about 5 Hz up to about 30 Hz, or up to about 40 Hz, or up to about 50 Hz.

52. The system according to any one of claims 50-51, wherein said system is configured to provide transcutaneous stimulation signal at an amplitude ranging from 10 mA to about 500 mA or up to about 300 mA, or up to about 150 mA, or from about 20 mA up to about 50 mA or up to about 100 mA, or from about 20 mA or from about 30 mA, or from about 40 mA up to about 50 mA, or up to about 60 mA, or up to about 70 mA, or up to about 80 mA.

53. The system according to any one of claims 50-52, wherein system is configured to provide a transcutaneous stimulation signal pulse width that ranges from about 100 μs up to about 1000 μs, or from about 150 μs up to about 600 μs, or from about 200 μs up to about 500 μs, or from about 200 μs up to about 450 μs.

54. The system according to any one of claims 50-53, wherein said system is configured to deliver said transcutaneous stimulation signal superimposed on a high frequency carrier signal.

55. The system of claim 54, wherein said high frequency carrier signal ranges from about 3 kHz, or about 5 kHz, or about 8 kHz up to about 100 kHz, or up to about 80 kHz, or up to about 50 kHz, or up to about 40 kHz, or up to about 30 kHz, or up to about 20 kHz, or up to about 15 kHz.

56. The system of claim 54, wherein said high frequency carrier signal is about 10 kHz.

57. The system according to any one of claims 54-56, wherein said carrier frequency amplitude ranges from about 30 mA, or about 40 mA, or about 50 mA, or about 60 mA, or about 70 mA, or about 80 mA up to about 500 mA, or up to about 400 mA, or up to about 300 mA, or up to about 200 mA, or up to about 150 mA.

58. The system according to any one of claims 50-57, wherein said system is configured to provide transcutaneous stimulation at a frequency and amplitude sufficient to stimulate and/or to improve postural and/or locomotor activity and/or postural or locomotor strength.

59. The system according to any one of claims 50-57, wherein said system is configured to provide transcutaneous stimulation at a frequency and amplitude sufficient to stimulate and/or to improve reaching and/or grasping and/or fine motor control of a hand.

60. The system according to any one of claims 50-57, wherein said system is configured to provide transcutaneous stimulation at a frequency and amplitude sufficient to stimulate voluntary voiding of the bladder and/or bowel, and/or return of sexual function, and/or autonomic control of cardiovascular function, and/or body temperature, control of digestive functions, control of kidney functions, chewing, swallowing, drinking, talking, or breathing.

61. The system according to any one of claims 50-57, wherein said system is configured to provide transcutaneous stimulation at a frequency and amplitude sufficient to stimulate voluntary voiding of the bladder and/or bowel, and/or return of sexual function, and/or autonomic control of cardiovascular function, and/or body temperature, control of digestive functions, control of kidney functions, chewing, swallowing, drinking, talking, or breathing.

62. A method of stimulating or improving postural and/or locomotor activity and/or postural or locomotor strength, and/or reaching or grasping, and/or fine motor control of a hand, and/or enabling one or more functions selected from the group consisting of voluntary voiding of the bladder and/or bowel, return of sexual function, autonomic control of cardiovascular function, and body temperature control, control of digestive functions, control of kidney functions, chewing, swallowing, drinking, talking, or breathing in a normal subject or a subject having a neurologically derived paralysis said method comprising neuromodulating the spinal cord of said subject or a region thereof by administering transcutaneous stimulation to the spinal cord or a region thereof using an electrical stimulator electrically coupled to a needle electrode according to any one of claims 1-36 or an electrode array according to any one of claims 37-49, wherein said needle electrode or at least a part of said electrode array is disposed on the skin surface over the spinal cord or a region thereof.

63. The method of claim 62, wherein said transcutaneous stimulation is at a frequency ranging from about 0.5 Hz or from about 3 Hz, or from about 5 Hz, or from about 10 Hz up to about 50 kHz, or up to about 30 kHz, or up to about 20 kHz, or up to about 10 kHz, or up to about 1,000 Hz, or up to about 500 Hz, or up to about 100 Hz, or up to about 80 Hz, or up to about 40 Hz, or from about 3 Hz or from about 5 Hz up to about 80 Hz, or from about 5 Hz up to about 30 Hz, or up to about 40 Hz, or up to about 50 Hz.

64. The method according to any one of claims 62-63, wherein said transcutaneous stimulation is at an amplitude ranging from 10 mA to about 500 mA, or up to about 300 mA, or up to about 150 mA, or from about 20 mA to about 300 mA, or up to about 50 mA or up to about 100 mA, or from about 20 mA or from about 30 mA, or from about 40 mA to about 50 mA, or to about 60 mA, or to about 70 mA or to about 80 mA.

65. The method according to any one of claims 62-64, wherein said transcutaneous stimulation pulse width ranges from about 100 μs up to about 1000 μs, or from about 150 μs up to about 600 μs, or from about 200 μs up to about 500 μs, or from about 200 μs to about 450 μs.

66. The method according to any one of claims 62-65, wherein said transcutaneous stimulation is superimposed on a high frequency carrier signal.

67. The method of claim 66, wherein said high frequency carrier signal ranges from 3 kHz, or about 5 kHz, or about 8 kHz up to about 100 kHz, or up to about 80 kHz, or up to about 50 kHz, or up to about 40 kHz, or up to about 30 kHz, or up to about 20 kHz, or up to about 15 kHz.

68. The method of claim 66, wherein said high frequency carrier signal is about 10 kHz.

69. The method according to any one of claims 66-68, wherein said carrier frequency amplitude ranges from about 30 mA, or about 40 mA, or about 50 mA, or about 60 mA, or about 70 mA, or about 80 mA up to about 500 mA, or up to about 300 mA, or up to about 200 mA, or up to about 150 mA.

70. The method according to any one of claims 62-69, wherein said transcutaneous stimulation is at a frequency and amplitude sufficient to stimulate and/or to improve postural and/or locomotor activity and/or postural or locomotor strength.

71. The method according to any one of claims 62-69, wherein said transcutaneous stimulation is at a frequency and amplitude sufficient to stimulate and/or improve reaching and/or grasping and/or fine motor control of a hand.

72. The method according to any one of claims 62-69, wherein said transcutaneous stimulation is at a frequency and amplitude sufficient to stimulate voluntary voiding of the bladder and/or bowel, and/or return of sexual function, and/or autonomic control of cardiovascular function, and/or body temperature, control of digestive functions, control of kidney functions, chewing, swallowing, drinking, talking, or breathing

73. The method according to any one of claims 62-72, wherein said transcutaneous stimulation is applied on the skin surface over the cervical spine or a region thereof and/or over the thoracic spine or a region thereof, and/or over the lumbosacral spine or a region thereof.

74. The method according to any one of claims 62-72, wherein said transcutaneous stimulation is applied on the skin surface over a region of the spinal cord that controls the lower limbs upper limbs to stimulate or improve postural and/or locomotor activity and/or postural or locomotor strength.

75. The method of claim 74, wherein said locomotor activity comprises standing and/or stepping.

76. The method of claim 74, wherein said locomotor activity comprises sitting down or laying down.

77. The method of claim 74, wherein said movement comprises stabilizing sitting or standing posture.

78. The method according to any one of claims 62-72, wherein said transcutaneous stimulation is applied on the skin surface over a region of the spinal cord that controls the upper limbs to improve reaching and/or grasping and/or to improve improving motor control and/or strength in a hand and/or upper limb of a subject with a neuromotor disorder affecting motor control of the hand and/or upper limb.

79. The method according to any one of claims 74-77, wherein said method comprises subjecting said subject to physical training that exposes said subject to relevant postural and locomotor or motor proprioceptive signals.

80. The method of claim 79, wherein the wherein the combination of said stimulation and physical training modulates in real time the electrophysiological properties of spinal circuits in said subject so they are activated by proprioceptive information derived from the region of the subject where said previously stated functions are facilitated.

81. The method according to any one of claims 79-80, wherein said physical training comprises inducing a load bearing positional change in the region of the subject where locomotor activity is to be facilitated.

82. The method according to claim 81, wherein the load bearing positional change in said subject comprises standing.

83. The method according to claim 81, wherein the load bearing positional change in said subject comprises stepping.

84. The method according to claim 81, wherein the load bearing positional change in said subject comprises reaching.

85. The method according to claim 81, wherein the load bearing positional change in said subject comprises grasping.

86. The method according to any one of claims 74-85, wherein said physical training comprises robotically guided training.

87. The method according to any one of claims 74-86, wherein said physical training comprises hand contraction and/or upper limb movements against a resistance.

88. The method according to any one of claims 74-86, wherein said physical training comprises tracing a displayed pattern by hand manipulation of a hand controller.

89. The method according to any one of claims 62-88, wherein said transcutaneous stimulation is applied over a region of the spinal cord that controls the bladder and/or bowel.

90. The method according to any one of claims 62-89, wherein one or more needle electrodes are stimulated in a monopolar configuration.

91. The method according to any one of claims 62-89, wherein one or more needle electrodes are stimulated in a bipolar configuration.

92. The method according to any one of claims 62-91, wherein said stimulation comprises tonic stimulation.

93. The method according to any one of claims 62-92, wherein said stimulation comprises simultaneous or sequential stimulation of different spinal cord regions.

94. The method according to any one of claims 62-93, wherein the stimulation pattern is under control of the subject.

95. The method according to any one of claims 62-94, wherein one or more needle electrodes is used to record an electrical potential.

96. The method according to any one of claims 62-95, wherein said subject is administered at least one monoaminergic agonist.

97. The method of claim 96, wherein said at least one monoaminergic agonist comprises an agent selected from the group consisting of a serotonergic drug, a dopaminergic drug, a noradrenergic drug, a GABAergic drug, and a glycinergic drug.

98. The method of claim 97, wherein said agent is selected from the group consisting of 8-hydroxy-2-(di-n-propylamino)tetralin (8-OH-DPAT), 4-(benzodioxan-5-yl)1-(indan-2-yl)piperazine (S15535), N-{2-[4-(2-methoxyphenyl)-1-piperazinyl]ethyl}-N-(2-pyridinyl)cyclo-hexanecarboxamide (WAY 100.635), Quipazine, Ketanserin, 4-amino-(6-chloro-2-pyridyl)-1 piperidine hydrochloride (SR 57227A), Ondanesetron, Buspirone, Methoxamine, Prazosin, Clonidine, Yohimbine, 6-chloro-1-phenyl-2,3,4,5-tetrahydro-1H-3-benzazepine-7,8-diol (SKF-81297), 7-chloro-3-methyl-1-phenyl-1,2,4,5-tetrahydro-3-benzazepin-8-ol (SCH-23390), Quinpirole, and Eticlopride.

99. The method of claim 97, wherein said monoaminergic agonist is buspirone.

100. The method according to any one of claims 62-99, wherein said subject is a human.

101. The method according to any one of claims 62-100, wherein said subject has a spinal cord injury.

102. The method of claim 101, wherein said spinal cord injury is clinically classified as motor complete.

103. The method of claim 101, wherein said spinal cord injury is clinically classified as motor incomplete.

104. The method according to any one of claims 62-100, wherein said subject has an ischemic brain injury.

105. The method of claim 104, wherein said ischemic brain injury is brain injury from stroke or acute trauma.

106. The method according to any one of claims 62-100, wherein said subject has a neurodegenerative pathology.

107. The method of claim 106, wherein said neurodegenerative pathology is associated with a condition selected from the group consisting of stroke, Parkinson's disease, Huntington's disease, Alzheimer's disease, amyotrophic lateral sclerosis (ALS), primary lateral sclerosis (PLS), dystonia, and cerebral palsy.
